(19) United States
(12) Patent Application Publication
(10) Pub. No.: US 2024/0271180 A1
(43) Pub. Date: Aug. 15, 2024

(54) METHOD FOR DETECTING CONTAMINATING LIPASE ACTIVITY

(71) Applicant: BOEHRINGER INGELHEIM INTERNATIONAL GMBH, Ingelheim am Rhein (DE)

(72) Inventors: Matthias Joseph KNAPE, Warthausen (DE); Melanie MILLER, Erolzheim (DE); Benjamin Joshua KOHLER, Ravensburg (DE); Oliver BURKERT, Mittelbiberach (DE)

(21) Appl. No.: 18/044,110

(22) PCT Filed: Sep. 6, 2021
(86) PCT No.: PCT/EP2021/074522

§ 371 Date: Mar. 6, 2023

(30) Foreign Application Priority Data
Sep. 7, 2020 (EP) 20194935.1
Dec. 2, 2020 (EP) 20211184.5

(51) Int. Cl. C12Q 1/44; G01N 21/64

(52) U.S. Cl. CPC C12Q 1/44; G01N 21/6428; C12Y 301/01003

(57) ABSTRACT

The present invention relates to a method for detecting contaminating lipase activity in a sample of a recombinant protein by measuring the hydrolysis of a substrate comprising the chromophore 4-methylumbelliferyl (4-MU) in the form of a 4-MU ester, wherein the 4-MU ester is a saturated unbranched-chain fatty acid (C6-C16) 4-MU ester. Further provided is a kit for determining contaminating lipase activity in a sample of a recombinant protein.

### CROSS-REFERENCE TO RELATED APPLICATIONS

This application is a National Stage Application of PCT Application Serial No. PCT/EP2021/074522, filed Sep. 6, 2021, which claims priority to European Application Nos. EP 20211184.5, filed Dec. 2, 2020, and EP 20194935.1, filed Sep. 7, 2020, the entire contents of the aforementioned applications are hereby incorporated herein by reference.

### FIELD OF THE INVENTION

The present invention relates to a method for detecting contaminating lipase activity in a sample of a recombinant protein. More specifically, the method comprises contacting at least one sample (such as an IPC sample) with a reaction solution comprising (i) a buffer having a pH of about pH 4 to about pH 9, (ii) a non-denaturing surfactant not having an ester-bond, wherein the surfactant is a non-ionic or zwitter-ionic surfactant, (iii) a substrate comprising the chromophore 4-methylumbelliferyl (4-MU) in the form of a 4-MU ester, wherein the 4-MU ester is a saturated unbranched-chain fatty acid (C6-C16) 4-MU ester, and (iv) optionally a non-buffering salt; and detecting contaminating lipase activity by measuring hydrolysis of the 4-MU ester and detecting the fluorescence intensity of the released chromophore 4-MU. Further provided is a kit for determining contaminating lipase activity in a sample comprising a recombinant protein, such as an IPC sample, comprising: (i) a buffer having a pH of about pH 4 to about pH 9, (ii) a non-denaturing surfactant not having an ester-bond, wherein the surfactant is a non-ionic or zwitter-ionic surfactant, and (iii) a substrate comprising the chromophore 4-methylumbelliferyl (4-MU) in the form of a 4-MU ester, wherein the substrate is a saturated unbranched-chain fatty acid (C6 to C16) 4-MU ester.

### BACKGROUND

Proteins as therapeutic agents have become increasingly popular in the last decades. Formulations comprising therapeutic proteins, such as monoclonal antibodies, often contain high protein concentration of 100 mg/ml or higher and often require the presence of a surfactant. The most widely used surfactants in biopharmaceutical industry due to their biocompatibility and low toxicity are polysorbates (PS), such as polysorbate 20 (polyoxyethylene (20) sorbitan monolaurate, Tween 20®) or polysorbate 80 (polyoxyethylene (20) sorbitan monooleate, Tween 80®).

Polysorbates are heterogeneous mixtures of sorbitol and its anhydrides along with approximately 20 polymerized ethylene oxide moieties partially esterified with fatty acids. However, polysorbates are prone to degradation, which can adversely affect product quality. Degradation may affect product quality not only due to the resulting reduced polysorbate concentration in the formulation, but also due to the formation of visible and sub-visible particles from insoluble matter of polysorbate degradants, such as fatty acids and polyoxyethylene side chains. Polysorbates can be degraded chemically or enzymatically. Chemical polysorbate degradation is mainly caused by an oxidative reaction causing the formation of inter alia aldehydes, ketones and fatty acids. Enzymatic polysorbate degradation is characterized by hydrolysis of the ester bond connecting the polyethoxylated sorbitan with the fatty acid (Dwivedi et al., 2018, International Journal of Pharmaceutics 552:442-436). Although oxidative degradation of polysorbates has been known for a long time, enzymatic hydrolysis of polysorbates in antibody formulations have only recently been considered as one of the major degradation pathways. In the recent years, polysorbate degradation has emerged as a major challenge in the biopharmaceutical community.

It has been reported that residual hydrolytic activity of lipases or other enzymes of host cell proteins (HCPs) contained in the final drug product (DP) can lead to polysorbate degradation. The role of lipases in the degradation of polysorbates in antibody formulations has further been emphasized by Chiu et al., wherein harvested cell culture fluid (HCCF) from lipoprotein lipase (LPL) knockout CHO cells reduced the PS20 and PS80 degradation as compared to wild type (Chui et al., 2017, Biotechnol. Bioeng. 114, 1006-1015). Necessary alterations and adaptations of the upstream and particularly downstream production processes in therapeutic protein production are difficult, as determining the impact of single purification steps and conditions on polysorbate degradation takes several weeks.

Polysorbate content and degradation can be studied using different analytical techniques. The most commonly used method for quantification of polysorbates is reverse phase liquid chromatography (such as RP-HPLC) and this may further be coupled to evaporative light scattering detector (ELSD) and charged aerosol detector (CAD). Other techniques capable of polysorbate content determination consists of fluorescence micelle assay (FMA) or a chemical complexation of the sorbitan ring with cobalt thiocyanate or ferric thiocyanate. However, in order to determine whether alterations in the purification process were successful in terms of reducing the hydrolytic activity responsible for polysorbate degradation, samples of interest need to be spiked with polysorbate and its degradation needs to be analyzed as described above. Thus, polysorbate degradation is typically assessed by monitoring the decrease of polysorbate content over time. However, polysorbate degradation is a slow process that may take up to several weeks or months. Further, the analytics are complex and time consuming.

In order to develop purification conditions that minimize enzymatic polysorbate degradation in the drug product, there is a need for a fast, reliable automated high-throughput assay with high sensitivity, which can be easily adapted to the different samples and provides predictive information about hydrolytic activity responsible for polysorbate degradation in a drug substance of drug product sample. Such an assay is useful as tool to guide process development for drug substance production with increased product quality due to minimized polysorbate degrading activity co-purified with the target protein.

Detection of hydrolytic activity of lipases in vitro using fluorescent substrates has been known in the art, but these prior art assays are not sufficiently sensitive to reliably detect within a short period of time contaminating lipase activity in a recombinant protein preparation, which has only been co-purified from eukaryotic cells with the recombinant protein (protein of interest). For example, Tsuzuki et al., (Biosci. Biotechnol. Biochem, 2001, 65(9): 2078-2082) analyse the activity of several lipases from microorganisms at high concentrations using fluorescent substrates and found that DMSO increases hydrolysis of strongly hydrophobic substrates for certain lipases. DMSO is an organic solvent regularly used to solubilize the substrate, which is not a surfactant forming micelles. Sulciene et al., (Acta Paediatrica, supplement, 2018, 116:1049-1055) discloses the use of immobilized lipolytic enzymes from yeast to produce epoxidized oils and describes detecting lipase activity of these concentrated lipase-nanoparticle conjugates using fluorescent substrate and without disclosing the exact conditions. Likewise, Yoo et al., (Cell Chemical Biology, 2020, 27: 143-157) discloses a fluorogenic substrate assay for detecting lipase activity and uses Triton X-100 for solubilizing the highly concentrated lipase rPfMAGLLP prior to analysis, but not as part of the reaction solution. WO 2010/024924 discloses an assay for screening for lipases expressed in E. coli using a fluorogenic substrate and hence again the assay is not used for detecting contaminating lipase activity in recombinant protein samples purified from eukaryotic cells. Yet none of these prior art assays determine contaminating lipase activity in a recombinant protein sample produced in eukaryotic cells.

Menden et al., 2019 (Journal of Enzyme Inhibition of Medicinal Chemistry, 34(1): 1474-1480) reports a lipase activity assay in which lipase activity of a defined enzyme extract of Candida rugosa lipase (CRL) isoforms is detected to verify the mode of action of the inhibitor tropolone using 4-methylumbelliferryl butyrate (4-MUB) and palmitate (4-MUP) as substrate. Limitations to the assay are reported, which include the intrinsic decrease in solubility of the hydrophobic fatty acid tail with length and the autocatalysis of the substrate in the basic pH range. Moreover, no surfactant is used in the assay. More recently, Jahn et al., 2020 (Pharm. Res. 37(118): 2-13) reports a chromophore-based lipase activity assay for use in determining polysorbate degradation in samples of harvested cell culture fluids using 4-methylumbelliferyl oleate (4-MuO) as substrate. However, the moderate sensitivity still requires incubation times of 24 hour or more.

Accordingly, there is still a need for an improved method with high sensitivity that can determine lipase activity in a relevant sample in a short period of time.

### SUMMARY OF THE INVENTION

The present invention relates to a method for detecting (contaminating) lipase activity in a sample comprising a recombinant protein comprising (a) providing at least one sample comprising a recombinant protein produced in a eukaryotic cell; (b) contacting the at least one sample with a reaction solution to form a reaction mixture, wherein the reaction solution comprises: (i) a buffer having a pH of about pH 4 to about pH 9, (ii) a non-denaturing surfactant not having an ester-bond, wherein the surfactant is a non-ionic or zwitter-ionic surfactant, (iii) a substrate comprising the chromophore 4-methylumbelliferyl (4-MU) in the form of a 4-MU ester, wherein the 4-MU ester is a saturated unbranched-chain fatty acid (C6-C16) 4-MU ester, and (iv) optionally a non-buffering salt; (c) incubating the sample and the substrate in the reaction mixture; and (d) detecting (contaminating) lipase activity by measuring hydrolysis of the 4-MU ester and detecting the fluorescence intensity of the released chromophore 4-MU (which is a 4-MU ester hydrolysis product); wherein optionally hydrolysis is measured by detecting the fluorescence intensity of the released chromophore 4-MU over time, while incubating the sample and the substrate in the reaction mixture according to step (c). The method is to be understood to refer to an in vitro method. In certain embodiments the sample and the substrate in the reaction mixture are incubated for any time period between 2 min and less than 5 hours, less than 3 hours, less than 2 hours, or less than 0.5 hours. The at least one sample may be a harvested cell culture fluid (HCCF), an in-process control (IPC) sample, a drug substance sample or a drug product sample. The recombinant protein in the sample for detecting lipase activity is preferably a therapeutic protein, such as an antibody, an antibody fragment, an antibody derived molecule or an fusion protein (e.g., an Fc fusion protein). According to the invention, the recombinant protein in the sample for detecting lipase activity is not a lipase and/or does not comprise lipase activity. Thus, any lipase activity detected in the at least one sample is contaminating lipase activity and/or derived from at least one contaminating protein having lipase activity, such as host cell proteins (HCPs) derived from the eukaryotic cell.

In a preferred embodiment the substrate is selected from the group consisting of 4-methylumbelliferyl octanoate, 4-methylumbelliferyl nonanoate, 4-methylumbelliferyl decanoate (4-MUD), 4-methylumbelliferyl undecanoate and 4-methylumbelliferyl dodecanoate, more preferably the substrate is selected from the group consisting of 4-methylumbelliferyl octanoate, 4-methylumbelliferyl decanoate (4-MUD) and 4-methylumbelliferyl dodecanoate. According to preferred embodiments of the invention, the surfactant has a final concentration in the reaction mixture above its critical micelle concentration in the reaction mixture. The non-denaturing non-ionic or zwitter-ionic surfactant may be CHAPS, CHAPSO, Zwittergent (such as Zwittergent 3-12) or a saponin. Preferably, the non-denaturing non-ionic or zwitter-ionic surfactant is CHAPS. CHAPS may be provided at a final concentration in the reaction mixture of about 8 mM to about 20 mM, preferably at about 8 mM to about 15 mM, more preferably at about 10 mM. A suitable buffer comprises one or more buffer substances selected from the group consisting of formic acid, acetic acid, lactic acid, citric acid, malic acid, maleic acid, glycine, glycylglycine, succinic acid, TES (2-{[tris(hydroxymethyl) methyl]amino}ethanesulfonic acid), MOPS (3-(N-morpholino)propanesulfonic acid), PIPES (piperazine-N,N′-bis(2-ethanesulfonic acid)), MES (2-(N-morpholino)ethanesulfonic acid), Tris base, Bis-Tris, Bis-Tris-Propane, Bicine (N, N-bis(2-hydroxyethyl)glycine), HEPES (4-2-hydroxyethyl-1-piperazineethanesulfonic acid), TAPS (3-([tris(hydroxymethyl)methyl]amino}pro-panesulfonic acid), Tricine (N-tris(hydroxymethyl)methylglycine), Na₂HPO₄ and NaH₂PO₄. Preferably, the buffer has a pH of about 5 to about 7.5, preferably the buffer has a pH of about 5.5 to about 7.5. In certain embodiments the buffer is a multi-component buffer having a buffering range from at least about pH 5 to at least about pH 7.5, preferably from at least about pH 4 to at least about pH 8.

The optional non-buffering salt may be, e.g., NaCl, KCl and CaCl₂) and is preferably NaCl or KCl. It may be provided at a concentration of about 100 mM to about 200 mM, preferably about 130 mM to about 170 mM, more preferably about 140 mM to about 150 mM in the reaction mixture. The ionic strength of non-buffering salt in the reaction mixture is preferably about 200 mM or less, more preferably about 170 mM or less and more preferably about 150 mM or less, such as from about 100 mM to about 200 mM, preferably about 130 mM to about 170 mM, more preferably about 140 mM to about 150 mM in the reaction mixture. Alternatively or in addition the cumulative ionic strength of the buffer and the non-buffering salt in the reaction mixture may be about 450, preferably about 400 mM or less, more preferably about 350 mM or less.

In another aspect, the invention relates to a method of manufacturing a recombinant protein of interest comprising the steps of (i) cultivating a eukaryotic cell expressing a recombinant protein of interest in cell culture; (ii) harvesting the recombinant protein; (iii) purifying the recombinant protein; and (iv) optionally formulating the recombinant protein into a pharmaceutically acceptable formulation suitable for administration; and (v) obtaining at least one sample comprising the recombinant protein in steps (ii), (iii) and/or (iv); wherein the method further comprises detecting (contaminating) lipase activity in a sample comprising the recombinant protein comprising: (a) providing the at least one sample comprising the recombinant protein produced in a eukaryotic cell of step (v); (b) contacting the at least one sample with a reaction solution to form a reaction mixture, wherein the reaction solution comprises: (i) a buffer having a pH of about pH 4 to about pH 9, (ii) a non-denaturing surfactant not having an ester-bond, wherein the surfactant is a non-ionic or zwitter-ionic surfactant, (iii) a substrate comprising the chromophore 4-methylumbelliferyl (4-MU) in the form of a 4-MU ester, wherein the 4-MU ester is a saturated unbranched-chain fatty acid (C6-C16) 4-MU ester, and (iv) optionally a non-buffering salt; (c) incubating the sample and the substrate in the reaction mixture; (d) detecting (contaminating) lipase activity by measuring hydrolysis of the 4-MU ester and detecting the fluorescence intensity of the released chromophore 4-MU; optionally measuring hydrolysis by detecting the fluorescence intensity of the released chromophore 4-MU over time, while incubating the sample and the substrate in the reaction mixture according to step (c). In certain embodiments, the method comprises obtaining at least one sample comprising the recombinant protein in step (ii), wherein the sample is a harvested cell culture fluid (HCCF) or a cell lysate; step (iii), wherein the sample is an in-process control (IPC) sample; and/or step (iv), wherein the sample is a drug substance sample or a drug product sample; preferably comprising obtaining at least one sample comprising the recombinant protein in step (iii), comprising obtaining at least one sample before and after affinity chromatography, and/or before and after acid treatment, before and after depth filtration following acid treatment, and/or before and after ion exchange chromatography, preferably anion exchange chromatography or cation exchange chromatography.

In another aspect the invention relates to a kit for determining contaminating lipase activity in a sample comprising a recombinant protein, such as an IPC sample, comprising: (i) a buffer having a pH of about pH 4 to about pH 9, (ii) a non-denaturing surfactant not having an ester-bond, wherein the surfactant is a non-ionic or zwitter-ionic surfactant, (iii) a substrate comprising the chromophore 4-methylumbelliferyl (4-MU) in the form of a 4-MU ester, wherein the substrate is a saturated unbranched-chain fatty acid (C6 to C16) 4-MU ester, and (iv) optionally a non-buffering salt, and/or (iiv) optionally water for dilution. In certain embodiments, the buffer, the surfactant and the optional non-buffering salt are premixed as an assay buffer. Preferably, said assay buffer is at least about 3-fold concentrated or about 3-fold to about 5-fold concentrated relative to a final reaction mixture. Alternatively, the assay buffer is provided as a dry mixture. Such dry mixture may be reconstituted with water to provide said at least about 3-fold concentrated or 5-fold concentrated assay buffer relative to a final reaction mixture. Alternatively or in addition the buffer, the surfactant, the substrate and the optional non-buffering salt are premixed and added as a master mix to the sample, wherein the master mix is provided at about 80% (v/v) to about 70% (v/v) of the reaction mixture, preferably at about 75% of the reaction mix.

In a preferred embodiment, the substrate is selected from the group consisting of 4-methylumbelliferyl octanoate, 4-methylumbelliferyl nonanoate, 4-methylumbelliferyl decanoate (4-MUD), methylumbelliferyl undecanoate and methylumbelliferyl dodecanoate. The kit may also further comprise an organic solvent for dissolving the substrate, or the substrate dissolved in an organic solvent, and/or one or more microtiter plate having 96 wells or a multiple of 96 wells. The non-denaturing non-ionic or zwitter-ionic surfactant may be CHAPS, CHAPSO, Zwittergent (such as Zwittergent 3-12) and a saponin. Preferably, the non-denaturing non-ionic or zwitter-ionic surfactant is CHAPS. In certain embodiments, the buffer comprises one or more buffer substances selected from the group consisting of formic acid, acetic acid, lactic acid, citric acid, malic acid, maleic acid, glycine, glycylglycine, succinic acid, TES (2-{[tris(hydroxyme-thyl)methyl]amino}ethanesulfonic acid), MOPS (3-(N-morpholino)propanesulfonic acid), PIPES (piperazine-N,N′-bis(2-ethanesulfonic acid)), MES (2-(N-morpholino)ethanesulfonic acid), Tris base, Tris, Bis-Tris, Bis-Tris-Propane, Bicine (N,N-bis(2-hydroxyethyl)glycine), HEPES (4-2-hydroxyethyl-1-piperazineethanesulfonic acid), TAPS (3-([tris(hydroxymethyl)methyl]amino-pro-panesulfonic acid), Tricine (N-tris(hydroxymethyl)methylglycine), Na₂HPO₄ and NaH₂PO₄. Preferably the buffer has a pH of about 5 to about 7.5, more preferably the buffer has a pH of about 5.5 to about 7.5. In certain embodiments the buffer is a multi-component buffer having a buffering range from at least about pH 5 to at least about pH 7.5, preferably from at least about pH 4 to at least about pH 8. The optional non-buffering salt may be, e.g., NaCl, KCl and CaCl₂) and is preferably NaCl or KCl.

### DESCRIPTION OF THE FIGURES

FIG. 1: Depicted is a hydrolysis reaction for the substrate 4-MUD.

FIG. 2: Detection of lipase activity in different bulk drug substances (BDS) at variable pH. Hydrolytic activity of bulk drug substances A-B and D-E (monoclonal antibodies and antibody-like formats) sample was measured at different pH between pH 4 and pH 8 as indicated. All measurements were performed with 30 μM 4-MUD in AMT assay buffer (75 mM acetate, 75 mM MES, 150 mM Tris, 150 mM NaCl, 10 mM CHAPS, pH 5.5) using a microplate reader (λₑₘ=450 nm, λₑₓ=330 nm, top read mode) in a black 96 well plate.

FIG. 3: Solubility of 4-MUD in the assay buffer. Solubility of 0 to 60 μM 4-MUD was tested with light scattering experiments in AMT assay buffer (75 mM acetate, 75 mM MES, 150 mM Tris, 150 mM NaCl, 10 mM CHAPS, pH 4-8) using a microplate reader (λₑₘ=450 nm, λₑₓ=330 nm, top read mode) in a black 96 well plate.

FIG. 4: Michaelis-Menten kinetics of the hydrolytic activity in a bulk drug substance (BDS D). Assay was performed in AMT assay buffer (75 mM acetate, 75 mM MES, 150 mM Tris, 150 mM NaCl, 10 mM CHAPS, pH 5.5) with varying concentrations of 4-MUD (1.5625 μM-150 μM) using a microplate reader (λₑₘ=450 nm, λₑₓ=330 nm, top read mode) in a black 96 well plate.

FIG. 5: Solubility of 4-MUD was tested with light scattering experiments in AMT assay buffer (75 mM acetate, 75 mM MES, 150 mM Tris, 150 mM NaCl, 20 mM CHAPS, pH 7) using a fluorescent spectrometer (λₑₘ=400 nm, λₑₓ=400 nm) in a 1 cm macro-cuvette.

FIG. 6: Autohydrolysis of 4-MUB and 4-MUD. Fluorescence was monitored for 1800 seconds with 30 UM of 4-MUB or 4-MUD, respectively in AMT assay buffer (75 mM acetate, 75 mM MES, 150 mM Tris, 150 mM NaCl, 10 mM CHAPS, pH 5.5) using a microplate reader (λₑₘ=450 nm, λₑₓ=330 nm, top read mode) in a black 96 well plate.

FIG. 7: Influence of CHAPS on lipase activity in bulk drug substances (BDS). (A) Hydrolytic activity of BDS G samples and buffer controls were measured with or without the addition of CHAPS and the released 4-MU in μM over time is provided. (B) Hydrolytic activity of BDS B, G & F (monoclonal antibodies and antibody-like formats) samples was measured with or without the addition of CHAPS. Activity of blank subtracted data normalized to the hydrolytic activity with CHAPS for each product is provided. Additionally, a representative IPC sample following ultrafiltration/diafiltration (UF/DF of Product D) is depicted. All measurements were performed with 30 μM 4-MUD in AMT assay buffer (75 mM acetate, 75 mM MES, 150 mM Tris, 150 mM NaCl, pH 5.5) with or without 10 mM CHAPS using a microplate reader (λₑₘ=450 nm, λₑₓ=330 nm, bottom read mode) in a black 96 well plate.

FIG. 8: Dependence of hydrolytic activity on ionic strength. Hydrolytic activity of an exemplary drug product (Product E) sample was measured in the presence of varying concentrations of NaCl (7.8125 mM-1000 mM). All measurements were performed with 30 PM 4-MUD in AMT assay buffer (75 mM acetate, 75 mM MES, 150 mM Tris, 10 mM CHAPS, pH 5.5) using a microplate reader (λₑₘ=450 nm, λₑₓ=330 nm, top read mode) in a black 96 well plate.

FIG. 9: Competitive inhibition of hydrolytic activity by PS20. Hydrolytic activity of a drug product sample was measured in the presence of varying concentrations of PS20 (0.0125 mg/ml-3.2 mg/mL). All measurements were performed with 30 μM 4-MUD in AMT assay buffer (50 mM acetate, 50 mM MES, 100 mM Tris, 150 mM NaCl, 10 mM CHAPS, pH 5.5) using a microplate reader (λₑₘ=450 nm, λₑₓ=330 nm, top read mode) in a black 96 well plate.

FIG. 10: Comparison of hydrolytic activity using CHAPS, Triton X-100 or Triton X 100 and gum arabicum in the assay buffer. Hydrolytic activity of PPL (A), bulk drug substance (BDS) B (B) and BDS E (C) at 0.024 mg/ml PPL or 2.4 mg/ml BDS in the reaction mixture was measured in standard conditions (AMT buffer, 5.5) with either 10 mM CHAPS (upper black line), 0.25% Triton X-100 (middle light grey line) or 0.25% Triton X-100 and 0.125% gum arabicum (lower dark grey line).

FIG. 11: Comparison of lipase activity in various IPC samples of one product (monoclonal antibody) measured with a fluorescence spectrometer (A) as well as a microplate reader (B). All measurements were performed with the phosphate assay buffer (81 mM Na₂HPO₄, 19 mM NaH₂PO₄, 140 mM NaCl, 10 mM CHAPS, pH 7.4) containing 3 μM 4-MUD. In the fluorescence spectrometer a 1 cm macro-cuvette and in the microplate reader a black 96 well plate (top read mode) has been used, both with λₑₘ=450 nm and λₑₓ=340 nm. The various IPC samples are indicated as follows: protein A elution buffer (Prot A buffer) and product pool (Prot A Prod P), neutralized acid treatment product pool (Neutral. AT Prod Pool), depth filtration product pool (Depth. filtr. Prod. P), cation exchange chromatography buffer (CIEX buffer) and product pool (CIEX Prod P), virus filtration product pool (Virus filtr. Prod P), 30 kD ultrafiltration/diafiltration buffer (UF/DF buffer) and product pool (UF/DF Prod P), formulation buffer, drug substance (DS).

FIG. 12: Inhibition of polysorbate degradation by Orlistat. A drug product sample (Product D) with 0.2 mg/mL PS20 was incubated at RT with several pull points up to 56 days. Residual PS20 content was measured using a HPLC-CAD method. 1 μM Orlistat resulted in a reduced degradation of PS20 compared to the control reaction (DMSO only).

FIG. 13: Inhibition of hydrolytic activity by Orlistat. Hydrolytic activity of a drug product sample (Product D) was measured in the presence of varying concentrations of Orlistat (7.3 nM-20 μM). All measurements were performed with 30 μM 4-MUD in AMT assay buffer (75 mM acetate, 75 mM MES, 150 mM Tris, 150 mM NaCl, 10 mM CHAPS, pH 5.5) using a microplate reader (λₑₘ=450 nm, λₑₓ=330 nm, top read mode) in a black 96 well plate. The results suggest that Orlistat is inhibiting hydrolytic activity responsible for polysorbate degradation as well as the hydrolytic activity monitored using the 4-MUD assay.

FIG. 14: Comparison of PS-degradation in PS-spiked IPC samples (Product B) and hydrolytic activity measured with the 4-MUD assay. PS degradation was determined using a FMA assay; Hydrolytic activity (4-MUD assay) was performed with the phosphate assay buffer (100 mM NaHPO₄, 140 mM NaCl, 10 mM CHAPS, pH 7.4) containing 3 μM 4-MUD using a fluorescence spectrometer (λₑₘ=450 nm, λₑₓ=340 nm) in a 1 cm macro-cuvette. The various IPC samples are indicated as follows: protein A column (MabSelect) depth filtration product (Cuno), cation exchange chromatography (Poros), bulk drug substance (BDS).

### DETAILED DESCRIPTION

The general embodiments “comprising” or “comprised” encompass the more specific embodiment “consisting of”. Furthermore, singular and plural forms are not used in a limiting way. As used herein, the singular forms “a”, “an” and “the” designate both the singular and the plural, unless expressly stated to designate the singular only.

The term “sample” as used herein refers to any sample comprising a recombinant protein, wherein the recombinant protein is produced in a eukaryotic cell in cell culture: The at least one sample may, e.g., be a harvested cell culture fluid (HCCF) or a cell lysate, an in-process control (IPC) sample, a drug substance (also referred to as bulk drug substance herein) sample or a drug product sample comprising a recombinant protein, such as an antibody, an antibody fragment, an antibody derived molecule or an fusion protein (e.g., an Fc fusion protein). As used herein, the recombinant protein comprised in the sample is not a lipase and/or does not comprise lipase activity. Thus, any lipase activity detected in the sample is contaminating lipase activity and/or derived from and at least one contaminating protein having lipase activity, such as host cell proteins (HCPs) derived from the eukaryotic cell.

The term “contaminating” or “contamination” as used herein refers to the presence of an undesired and/or unintentional substance, such as lipolytic activities accompanying host cell proteins and/or at least one protein or substance having a hydrolytic activity, such as a lipase activity, which can be regarded only as a trace component in comparison to other predominantly produced substances like proteins of interest with non-lipolytic activity or of proteins for medical treatments such as antibodies or antibody-like compounds. In the context of the present invention, a hydrolytic and particularly a lipase activity is undesired due to its polysorbate degrading potential that may be co-purified with the recombinant protein. This applies especially to finally formulated protein preparations which advantageously comprise such unwanted factors only to less than 1% (w/w), preferably less than 0.1% (w/w), more preferably less than 0.01% (w/w) in comparison to total protein content.

The term “lipase activity”, when used herein, refers to the activity of a substance, typically a protein (enzyme) that catalyzes the hydrolysis of an ester bond in lipids, such as fatty acid esters. A lipase is a hydrolase enzyme that splits esters into an acid and an alcohol in a chemical reaction with water, also referred to as hydrolysis. A lipase may be e.g., carboxylic ester hydrolases (EC 3.1.1), such as a carboxylesterase (EC 3.1.1.1), a triacylglycerol lipase (EC 3.1.1.3), a phospholipase A2 (EC 3.1.1.4), a lysophospholipase (EC 3.1.1.5), an (EC 3.1.1.23), galactolipase (EC 3.1.1.26), phospholipase A1 (EC 3.1.1.32), lipoprotein lipase (EC 3.1.1.34) or hormone-sensitive lipase (EC 3.1.1.79); a phosphoric diester hydrolase (EC 3.1.4) such as phospholipase D (EC 3.1.4.4), a phosphoinositide phospholipase C (EC 3.1.4.11), glycosylphosphatidylinositol phospholipase D (EC 3.1.4.50) or N-acetylphosphatidylethanolamine-hydrolysing phospholipase D (EC 3.1.4.54); or glycosphingolipid deacylase (EC 3.5.1.69).

The term “protein” is used interchangeably with “amino acid sequence” or “polypeptide” and refers to polymers of amino acids of any length. These terms also include proteins that are post-translationally modified through reactions that include, but are not limited to, glycosylation, acetylation, phosphorylation, glycation or protein processing. Modifications and changes, for example fusions to other proteins, amino acid sequence substitutions, deletions or insertions, can be made in the structure of a polypeptide while the molecule maintains its biological functional activity. For example, certain amino acid sequence substitutions can be made in a polypeptide or its underlying nucleic acid coding sequence and a protein can be obtained with the same properties.

The term “recombinant protein” as used herein relates to a protein generated by recombinant techniques, such as molecular cloning and may also be referred to as recombinant protein of interest. As used herein, the recombinant protein is the protein of interest, e.g., in a sample to be purified. Such methods bring together genetic material from multiple sources or create sequences that do not naturally exist. A recombinant protein is typically based on a sequence from a different cell or organism or a different species from the recipient host cell used for production of the protein in cell culture, e.g., a CHO cell or a HEK 293 cell, or is based on an artificial sequence, such as a fusion protein. In the context of the present invention the recombinant protein is the protein of interest, preferably a therapeutic protein, such as an antibody, an antibody fragment, an antibody derived molecule (e.g., scFv, bi- or multi-specific antibodies) or a fusion protein (e.g., an Fc fusion protein). Thus, in one embodiment the recombinant protein is selected from the group consisting of an antibody, an antibody fragment, an antibody derived molecule and a fusion protein.

The term “eukaryotic cell” as used herein refers to cells that have a nucleus within a nuclear envelop and include animal cells, human cells, plant cells and yeast cells. In the present invention a “eukaryotic cell” particularly encompasses mammalian cell, such as Chinese hamster ovary (CHO) cell or HEK293 cell derived cells, and yeast cells.

The term “drug substance (DS)” or “bulk drug substance (BDS)” is used synonymously herein and refers to the formulated active pharmaceutical ingredient (API) with excipients. The API has the therapeutic effect in the body as opposed to the excipients, which assist with the delivery of the API. In the case of biologic therapeutics, the formulated API with excipients typically means the API in the final formulation buffer at a concentration of at least the highest concentration used in the final dosage form, also referred to as drug product.

The term “drug product”, abbreviated as DP, as used herein refers to the final marketed dosage form of the drug substance for example a tablet or capsule or in the case of biologics typically the solution for injection in the appropriate containment, such as a vial or syringe. The drug product may also be in a lyophilized form.

The term “polysorbate 20” as used herein refers to a non-ionic polysorbate-type surfactant derived from polyethoxylated sorbitan and lauric acid (polyoxyethylene (20) sorbitan monolaurate). It is also known as Tween 20. Its stability and relative non-toxicity allow it to be used as a surfactant and emulsifier in a number of domestic, scientific analyses. Polysorbate 20 can be used as washing agent in immunoassays, Western blots and ELISA. It can further be used in pharmacological applications, such as pharmaceutical formulations, particularly for biologics, such as antibodies and Fc-fusion proteins. Particularly it helps to prevent non-specific antibody binding.

The term “polysorbate 80” as used herein refers to a non-ionic polysorbate-type surfactant derived from polyethoxylated sorbitan and oleic acid (polyoxyethylene (20) sorbitan monooleate). It is also known as Tween 80 and has a similar use as polysorbate 20.

The term “therapeutic protein” as used herein refers to proteins that can be used in medical treatment of humans and/or animals. These include, but are not limited to antibodies, growth factors, blood coagulation factors, vaccines, interferons, hormones and fusion proteins.

The term “produced” as used herein relates to the production of the recombinant protein, preferably a therapeutic protein, in a eukaryotic cell, preferably a yeast cell or a mammalian cell, in cell culture. The person skilled in the art knows how to produce recombinant proteins in cells using fermentation. The production of recombinant proteins comprises cultivating the eukaryotic cell expressing the recombinant protein of interest in cell culture. Cultivating the eukaryotic cell expressing the recombinant protein in cell culture comprises maintaining the eukaryotic cells in a suitable medium and under conditions that allow growth and/or protein production/expression. The recombinant protein may be produced by fed-batch or continuous cell culture. Thus, the eukaryotic cells may be cultivated in a fed-batch or continuous cell culture or a combination thereof, preferably in a fed-batch cell culture.

The term “expressing a recombinant protein” as used herein refers to a cell comprising a DNA sequence coding for the recombinant protein, which is transcribed and translated into the protein sequence including post-translational modifications, i.e., resulting in the production of the recombinant protein in cell culture.

The term “about” as used herein refers to a variation of 10% of the value specified, for example, about 50% carries a variation from 45 to 55%.

### A Method for Detecting Lipase Activity

The present invention relates to an (in vitro) method for detecting lipase activity in a sample comprising a recombinant protein comprising (a) providing at least one sample comprising a recombinant protein produced in a eukaryotic cell; (b) contacting the at least one sample with a reaction solution to form a reaction mixture, wherein the reaction solution comprises: (i) a buffer having a pH of about pH 4 to about pH 9, (ii) a non-denaturing surfactant not having an ester-bond, wherein the surfactant is a non-ionic or zwitter-ionic surfactant, (iii) a substrate comprising the chromophore 4-methylumbelliferyl (4-MU) in the form of a 4-MU ester, wherein the 4-MU ester is a saturated unbranched-chain fatty acid (C6-C16) 4-MU ester, and (iv) optionally a non-buffering salt; (c) incubating the sample and the substrate in the reaction mixture; (d) detecting lipase activity by measuring hydrolysis of the 4-MU ester (the substrate) and detecting the fluorescence intensity of the released chromophore 4-MU (which is a 4-MU ester hydrolysis product); optionally measuring hydrolysis by detecting the fluorescence intensity of the released chromophore 4-MU over time, while incubating the sample and the substrate in the reaction mixture according to step (c). The method may further comprise a step of analyzing the data obtained from measuring hydrolysis for the at least one sample. The reaction solution used in the method of the invention is an aqueous reaction solution. The person skilled in the art will also understand that the at least one sample comprises a recombinant protein produced in a eukaryotic cell in cell culture. Further, the method according to the invention is for detecting contaminating lipase activity and the lipase activity detected in step (d) is contaminating lipase activity in the at least one sample comprising the recombinant protein, more specifically the recombinant protein of interest.

The assay read out may be as fast as 20 min or even faster. Thus, in certain embodiments the sample and the substrate in the reaction mixture are incubated for less than 5 hours, less than 3 hours, less than 2 hours, or less than 0.5 hours. For obtaining enough data points it is advisable to incubate the sample and the substrate in the reaction mixture for at least 1 min, at least 2 min or at least 5 min. Thus, the sample and the substrate in the reaction mixture may be incubated for any time period between 2 min and less than 5 hours, less than 3 hours, less than 2 hours, or less than 0.5 hours. The at least one sample may be a HCCF, an in-process control (IPC) sample, a drug substance or a drug product. According to the invention the recombinant protein in the sample is not a lipase and/or does not comprise lipase activity. Moreover, the recombinant protein in the sample according to the methods of the present invention is not an esterase or hydrolase and/or does not comprise an esterase or hydrolase activity. Thus, any lipase activity detected in the at least one sample is contaminating lipase activity and/or derived from at least one contaminating protein having lipase activity, such as one or more host cell proteins (HCPs) derived from the eukaryotic cell. The at least one sample comprising a recombinant protein produced in a eukaryotic cell may therefore potentially further comprises at least one contaminating protein having lipase activity. In one embodiment, the method comprises in step (a) providing at least one sample comprising a recombinant protein produced in a eukaryotic, preferably mammalian cell, in cell culture and host cell proteins (HCPs); and detecting in step (d) the lipase activity of said HCPs by measuring hydrolysis of the 4-MU ester and detecting the fluorescence intensity of the released chromophore 4-MU.

The substrate comprising the chromophore 4-MU in the form of saturated unbranched-chain fatty acid (C6 to C16) 4-MU ester, wherein the acyl chain of the saturated unbranched-chain fatty acid has from C6 to C16 carbon atoms. This substrate mimics the critical ester bond of polysorbate, i.e., a fatty acid ester bond.

Hydrolysis may be stopped at certain time points prior to detection of the fluorescence intensity of the released chromophore 4-MU. Alternatively and preferably, the fluorescence intensity of the released chromophore 4-MU may be detected in real-time without stopping hydrolysis of the 4-MU ester. In certain embodiments, the fluorescence intensity of the released chromophore 4-MU is detected without stopping hydrolysis of the 4-MU ester. In certain embodiments hydrolysis is measured by detecting the fluorescence intensity of the released chromophore 4-MU over time, while incubating the sample and the substrate in the reaction mixture according to step (c).

Real-time detection allows measuring hydrolysis over time and hence the specific reaction rate may be determined. In the method according to the invention hydrolysis of 4-MU ester in the reaction mixture typically follows a pseudo-zero order reaction rate. Detecting fluorescence in real-time therefore allows measurement in a time-frame with a pseudo-zero order reaction rate. Thus, in certain embodiments, the fluorescence intensity of the released chromophore 4-MU is detected over time and follows a pseudo-zero order reaction rate. Optionally a reaction mixture that does not meet the requirement of a pseudo-zero order reaction rate is excluded from analysis. A pseudo-zero order reaction rate can be assessed by linear regression analysis. Preferably, samples are run at least in triplicates and individual reaction mixtures are excluded from analysis in case they do not meet a pseudo-zero order reaction rate, e.g. due to bubbles in the well etc., to eliminate outliers. Eliminating outliers as described strongly increases sensitivity of the assay. Calibration curves using defined concentrations of 4-MU can be used to calculate the rate of hydrolysis (e.g. nmol/s). Calibration curves with known 4-MU concentrations further allow the determination and comparison of reaction velocities at different pH values.

The term “reaction rate” as used herein refers to the velocity of an enzyme converting a substrate into at least one product within a specific period. In some reactions, the rate is apparently independent of the reactant concentration. This means that the rate of the equation is equal to the rate constant, k, of the reaction and is referred to as zero-order reaction. A zero-order kinetics is always an artefact of the conditions under which the reaction is carried out. For this reason, reactions that follow zero-order kinetics are often referred to as pseudo-zero-order reactions.

The method according to the invention may further comprise a step of determining the rate of hydrolysis by detecting the fluorescence intensity of the released chromophore 4-MU as relative fluorescent units (RFU) and determining the amount of the released chromophore 4-MU (mol/s) by comparing it to a calibration curve generated by using defined concentrations of 4-MU. Typically, activity is measured by release of 4-MU in nmol/min. Alternatively or in addition a relative value may be calculated compared to an internal standard, such as another sample or preferably a commercially available lipase such as porcine pancreatic lipase (PPL) that serves as a positive control.

Incubation of the sample and the substrate in the reaction mixture allows the potentially present at least one contaminating protein having lipase activity to hydrolyze the 4-MU ester. Incubation is typically from a few minutes to a few hours. In one embodiment hydrolysis is measured by detecting the fluorescence intensity of the released chromophore 4-MU over time, while incubating the sample and the substrate in the reaction mixture according to step (c), i.e., in real-time during incubation. Due to the sensitivity of the assay, detection typically starts immediately following step (b). Incubation and hence detection time may depend on the lipase activity present in the sample and does typically not exceed 5 hours, preferably not 3 hours. In certain embodiments, the sample and the substrate in the reaction mixture are incubated for less than 5 hours, less than 3 hours, less than 2 hours, less than 1 hour, or less than 0.5 hours. For obtaining enough data points it is advisable to incubate the sample and the substrate in the reaction mixture for at least about 1 min, at least about 2 min or at least about 5 min. Thus, the sample and the substrate in the reaction mixture may be incubated for any time period between about 2 min and less than 5 hours, less than 3 hours, less than 2 hours, or less than 0.5 hours. Preferably, the sample and the substrate in the reaction mixture are incubated between 20 minutes and 2 hours at a temperature of about 25° C. Since reaction temperature influences reaction time, the reaction temperature should be kept constant during measurement, such as at a constant temperature between 20-37ºC, preferably between 22-28° C., more preferably between 24-26° C. In one embodiment, the sample and the substrate in the reaction mixture are incubated for less than 5 hours, less than 3 hours, less than 2 hours or less than 1 hour at a constant temperature between 20-37° C., preferably between 22-28° C., more preferably between 24-26° C. or for any time period between about 2 min and less than 5 hours, less than 3 hours, less than 2 hours or less than 1 hour at a constant temperature between 20-37° C., preferably between 22-28° C., more preferably between 24-26° C.

The substrate comprising the chromophore 4-MU is in the form of saturated unbranched-chain fatty acid (C6 to C16) 4-MU ester, wherein the acyl chain of the saturated unbranched-chain fatty acid has from C6 to C16 carbon atoms. This substrate mimics key feature of polysorbate, i.e., a fatty acid ester bond and a long acyl chain. Polysorbate 20 is an ester of the fatty acid lauric acid, a saturated unbranched-chain fatty acid. Polysorbate 80 in comparison is an ester of the fatty acid oleic acid, an unsaturated fatty acid.

Unsaturated fatty acids are more bulky than saturated fatty acids due to the double bond(s) and further branched-chain fatty acids are more bulky compared to unbranched-chain fatty acids. Lipase activity in a sample comprising a recombinant protein may be mediated by one or more lipases or other hydrolyzing enzymes and differ between various products, such as individual antibodies (see FIG. 2). Thus, in most cases the contaminating protein(s) with lipase activity is/are unknown and may be a mixture of more than one protein. Many lipases, such as triacylglycerol lipases, can be in an open state or in a closed state whereas the active site is shielded from the solvent by a part of the polypeptide chain, the flap or lid. Thus, the active site of many lipases resembles a cavity or the inside of a barrel, which most likely determines substrate specificity. An ester of a saturated unbranched-chain fatty acid (less bulky fatty acid) that is of medium length is therefore likely to capture a broader enzyme spectrum compared to, e.g., oleate having a longer and unsaturated acyl chain as used in the method of the invention. Preferable the substrate captures an equal or broader enzyme spectrum compared to PS20 or PS80.

Further, fatty acid esters with shorter acyl chains offer better solubility in water-based reaction mixtures compared to longer chain length fatty acid esters. Consequently, more substrate can be used in the assay mix. More specifically, it was found that solubility becomes strongly limiting at a chain length of C16 or longer.

Additionally, it was found that the decanoate ester (4-MUD) offers a better resistance to auto-hydrolysis compared to e.g. a butyrate ester (4-MUB). It was found that a chain length up to C5 strongly increased auto-hydrolysis. The C10 fatty acid in 4-MUD was found to be optimal for use in the examples, but slightly longer or shorter saturated unbranched fatty acid esters, such as saturated unbranched-chain fatty acid (C6 to C16) 4-MU ester or more preferably saturated unbranched-chain fatty acid (C8 to C12) 4-MU ester may similarly be used in the method according to the invention. Thus, the 4-MU ester used in the method according to the invention has an acyl chain of the saturated unbranched-chain fatty acid from C6 to C16 carbon atoms. More preferably, the fatty acid is a medium-chain fatty acid and the 4-MU ester is a saturated unbranched-chain fatty acid (C8 to C12) 4-MU ester. In certain embodiments, the substrate is selected from the group consisting of 4-methylumbelliferyl octanoate, 4-methylumbelliferyl nonanoate, 4-methylumbelliferyl decanoate (4-MUD), 4-methylumbelliferyl undecanoate and 4-methylumbelliferyl dodecanoate. In certain preferred embodiments the substrate is selected from the group consisting of 4-methylumbelliferyl octanoate, 4-methylumbelliferyl decanoate (4-MUD) and 4-methylumbelliferyl dodecanoate. In a more preferred embodiment, the substrate is 4-MUD. The substrate is typically dissolved as a stock solution (such as a 100× stock solution relative to the concentration in the reaction mixture) in an organic solvent, such as dimethyl sulfoxide (DMSO) or dimethyl formamide (DMF), preferably DMSO. In certain embodiments the substrate is provided a stock solution dissolved in an organic solvent selected from DMSO or DMF, preferably DMF.

Suitable substrate concentration in the present invention may be about 1 μM to about 1 mM. Thus, in certain embodiments the substrate is provided at a final concentration in the reaction mixture of about 1 μM to about 1 mM, preferably about 1 μM to 300 μM, preferably 1 μM to 30 μM, more preferably about 3 M to 30 μM. In certain embodiments the substrate is provided as stock solution in an organic solvent, wherein the stock solution is added at about 1% to about 5% (v/v) of the reaction mix.

The method according to the invention comprises contacting the at least one sample with a reaction solution comprising a non-denaturing surfactant not having an ester-bond, wherein the surfactant is non-ionic or zwitter-ionic surfactant (also referred to herein as “non-denaturing non-ionic or zwitter-ionic surfactant not having an ester-bond”). The term “surfactant” as used herein refers to a surface-active compound that is able to form micelles and that lowers the surface tension between two liquids, between a gas and a liquid and between a liquid and a solid. A surfactant may also be referred to as a detergent herein. Surfactants are amphiphilic, i.e., comprising both hydrophobic groups (tail) and hydrophilic groups (head). Surfactants are typically organic compounds. In aqueous phase, surfactants form aggregates, such as micelles, where the hydrophobic tail forms the core of the aggregate and the hydrophilic heads are in contact with the surrounding aqueous liquid. The hydrophobic tail (also referred to as hydrophobic hydrocarbon moiety) therefore has a certain length to form micelles. Thus, surfactants as used herein do not encompass organic solvents, such as ethanol or dimethylsulfoxid (DMSO). The tail of most surfactants typically consists of one or more hydrocarbon chain, which can be branched, linear or aromatic. The surfactant may comprise one or more hydrophobic tail, preferably the surfactant comprises one hydrophobic chain (single-tailed surfactant). Surfactants are commonly classified according to the hydrophilic head group. A non-ionic surfactant has no charged groups in their head, an ionic surfactant carries a net positive (cationic), or negative (anionic) charge, and a zwitterionic surfactant contains two oppositely charged groups. Thus, non-ionic or zwitterionic surfactants do not carry a net charge at the hydrophilic head group and are therefore milder in nature. Moreover, in many surfactants the hydrophobic tail is linked to the hydrophilic head via an ester bond, as in PS20 or PS80. Moreover, the non-ionic or zwitterionic surfactant is a non-denaturing surfactant. The term “non-denaturing surfactant” as used herein refers to the effect of the surfactant with respect to protein structure. A non-denaturing surfactant does not disrupt protein-protein interactions, particularly of water-soluble proteins.

Surfactants comprising an ester bond are potential substrates to lipases and may therefore interfere with the assay. Moreover, denaturation of the proteins with lipase activity and hence interference with the lipase activity in the sample is to be avoided. The surfactant to be used in the method according to the invention is therefore a non-denaturing surfactant not having an ester-bond, wherein the surfactant is a non-ionic or zwitter ionic surfactant. Examples for suitable non-denaturing zwitter-ionic surfactants are without being limited thereto 3-[(3-cholamidopropyl)dimethylammonio]-1-propanesulfonate (CHAPS), 3-([3-Cholamidopropyl)dimethylammonio]-2-hydroxy-1-propanesulfonate (CHAPSO), CHAPS analogs (such as Big CHAP N,N-bis-(3-D-gluconamidopropyl)deoxycholamide), Zwittergent (different lengths, such as n-Dodecyl-N,N-dimethyl-3-ammonio-1-propanesulfonate (Zwittergent 3-12)) and 3-[N, N-Dimethyl(3-palmitoylaminopropyl)ammonio]-propanesulfonate or other amidosulfobetaine detergents. Examples for suitable non-denaturing, non-ionic surfactants are without being limited thereto pyranoside surfactants (such as Octyl β-D-glucopyranoside (OGP), Nonyl β-D-glucopyranoside, Dodecyl ß-D-maltopyranoside (DDM) or Octyl β-D-thioglucopyranoside), polyoxyethylene (23) lauryl ether (Brij 35) or other Polyoxyethylene ether; saponins (e.g. Digitonin), octylphenoxy polyethoxyethanol (IGEPAL CA-630), poloxamer 188, 338, 407 or tergitol. In certain embodiments, the non-denaturing surfactant (non-ionic or zwitter-ionic surfactant) not having an ester-bond is not an ethoxylate and/or does not comprise a polyethylene glycol group and/or does not comprise an aromatic ring. In certain embodiments, the non-denaturing non-ionic or zwitter-ionic surfactant not having an ester-bond is not an octoxinol-9, specifically not polyethylene glycol tert-octylphenyl ether (Triton X-100, CAS No. 9002-93-1) and/or polyethylene glycol nonylphenyl ether (NP-40, CAS No. 9016-45-9). In preferred embodiments, the surfactant is a non-denaturing surfactant not having an ester-bond, wherein the surfactant is a non-ionic or zwitter-ionic surfactant, more preferably the surfactant is a non-denaturing surfactant (non-ionic or zwitter-ionic surfactant) selected from the group consisting of CHAPS (CAS No. 75621-03-3 or its hydrate CAS No. 331717-45-4), CHAPSO (CAS No. 82473-24-3), Zwittergent (such as Zwittergent 3-12; CAS No. 14933-08-5) and a saponin (CAS No. 8047-15-2), preferably CHAPS. None of these exemplary suitable surfactants exhibit an ester bond or an acyl chain and are therefore not a substrate for lipases. These surfactants do not compete with the substrate and hence does not affect sensitivity of the assay. Additionally, the presence of a surfactant mediates solubility of the substrate at the used concentrations in water. The person skilled in the art would know how to identify further suitable non-denaturing non-ionic or zwitter-ionic surfactant not having an ester-bond by determining its effect on 4-MU ester hydrolysis under assay conditions, as e.g., demonstrated for PS20 in Example 7.

It has been shown that the presence of a surfactant (such as 10 mM CHAPS) increases lipase activity and hence improves sensitivity of the assay. Without being bound by theory it is hypothesized that a surfactant creates an environment that promotes lipase activity by allowing the rearrangement and opening of the lid or flap, which has been described to cover the active site (Grochulski P, Li Y, Schrag J D, et al. Protein Sci 1994; 3:82-91 and Grochulski P, Bouthillier F, Kazlauskas R J, et al. Biochemistry 1994; 33:3494-500). To achieve this effect, the surfactant should be above its critical micelle concentration (CMC).

Thus, according to the invention the non-denaturing surfactant (non-ionic or zwitter-ionic) has a final concentration in the reaction mixture above its critical micelle concentration (CMC) in the reaction mixture. CMC represents an important physicochemical characteristic of a given surfactant in aqueous solution. Micelles are spherical aggregates whose hydrocarbon groups are to a large extent out of contact with water. The term “critical micelle concentration” or “CMC” as used herein refers to the concentration of a surfactant above which micelles are formed (i.e., the maximum monomer concentration) and may be determined according to methods known in the art. For example, a suitable method for determining the CMC is the fluorescence micelle assay (FMA), which uses the partitioning of the fluorescent hydrophobic dye N-phenyl-1-napthylamine (NPN) into surfactant micelles. NPN exhibits a low-fluorescence quantum yield in aqueous environments, which increase in more hydrophobic environments such as the core of the micelles. This assay has originally been developed for CMC determination and has also been used to determine the content of polysorbate in biopharmaceuticals as in the examples. An alternative method utilizing enhancement of 1,6-diphenyl-1,3,5-hexatriene (DPH) fluorescence upon micellization is described by Chattopadhyay and Harikumar (FEBS Letters 391 (1996) 199-202).

The CMC for a surfactant is derivable from literature and is e.g., about 6 mM for CHAPS, about 8 mM for CHAPSO, about 2-4 mM for Zwittergent 3-12. In certain embodiments, the non-denaturing zwitter-ionic surfactant is CHAPS and is provided at a final concentration in the reaction mixture of about 8 mM to about 20 mM, preferably at about 8 mM to about 15 mM, more preferably at about 10 mM. In other embodiments the non-denaturing zwitter-ionic surfactant is CHAPSO and is provided at a final concentration in the reaction mixture of about 10 mM to about 20 mM, preferably at about 10 mM to about 15 mM. In yet another embodiment the non-denaturing zwitter-ionic surfactant is Zwittergent 3-12 and is provided at a final concentration in the reaction mixture of about 4 mM to about mM, preferably at about 6 mM to about 8 mM. In yet another embodiment the non-denaturing non-ionic surfactant is a saponin and is provided at a final concentration in the reaction mixture of about 0.001% to 0.01% (w/v).

The reaction solution used in the method according to the invention further comprises a buffer having a pH of about pH 4 to about pH 9. Preferably the method is performed using a buffer having a pH of about pH 4 to about pH 8, preferably about pH 5 to about pH 7.5, more preferably about pH 5.5 to about pH 7.5. The person skilled in the art will understand that the pH of the buffer is within its buffering range when used in the method of the invention. In principle any buffer known in the art can be used, provided that is has a buffering range within about pH 4 to about pH 9. The buffer may comprise a single buffer substance or may be a multiple component buffer. Multiple component buffers typically have a broader buffering range. For example the buffer may comprise one or more buffer substances selected from the group consisting of a formic acid, acetic acid, lactic acid, citric acid, malic acid, maleic acid, glycine, glycylglycine, succinic acid, TES (2-{[tris(hydroxymethyl)methyl]amino}ethanesulfonic acid), MOPS (3-(N-morpholino)propanesulfonic acid), PIPES (piperazine-N,N′-bis(2-ethanesulfonic acid)), MES (2-(N-morpholino)ethanesulfonic acid), Tris base, Tris, Bis-Tris, Bis-Tris-Propane, Bicine (N, N-bis(2-hydroxyethyl)glycine), HEPES (4-2-hydroxyethyl-1-piperazineethanesulfonic acid), TAPS (3-([tris(hydroxymethyl)methyl]amino}pro-panesulfonic acid), Tricine (N-tris(hydroxymethyl)methylglycine), Na₂HPO₄ and NaH₂PO₄. Preferably the buffer is a phosphate buffer (Na₂HPO₄ and NaH₂PO₄), a Tris buffer or a HEPES buffer. In certain embodiments, the buffer has a concentration of about 50 to 400 mM, preferably about 50 to 300 mM more preferably about 50 to 200 mM.

The buffer may further be a multi-component buffer comprising more than one buffer substance with overlapping buffering ranges in order to have a broader buffering range. The buffer may, e.g., comprise two, three, four, five or more buffering substances, preferably two or more buffering substances, more preferably three or more buffering substances. For example, the multi-component buffer may comprise two to four buffering substances, three to four buffering substances, more preferably 3 buffering substances. In certain embodiments, the multi-component buffer comprises at least three buffer substances with overlapping buffering ranges, preferably comprising at least one of Tris, MES and/or acetic acid, preferably acetic acid, MES and Tris at a ratio of 1:1:2.

Since the assay turned out to be sensitive to ionic strength it is important for the design of a suitable multi-component buffer not only that it comprises buffer substances with overlapping buffer ranges, but that the buffer only moderately changes (less than 15% preferably even less than 10%) ionic strength at different pH (range pH 4-8) (Ellis K J, Morrisson J F, 1982. Methods in Enzymology, 87: 405-426). For example, the AMT buffer comprising acetic acid, MES and Tris allows the use of the buffer at different pH with only moderately affecting ionic strength, e.g., to identify conditions, including pH conditions that reduce hydrolytic activity. This buffer further allows taking measurements at the pH of the sample to determine lipase activity at the specific conditions present in a sample as well as to compare lipase activity at different states during purification. The assay allows to further increase sensitivity by measuring the sample at pH optimum.

Thus, a multi-component buffer as disclosed herein allows for the use of a buffer with a variable pH from at least about pH 4 to at least about pH 8 or at least about pH 4 to at least about pH 9. Alternatively or in addition, the use of a buffer with different pH values between about pH 4 and about pH 9 affects the ionic strength of the buffer by less than 15%, preferably less than 10% or even less than 7.5% or less than 5%, such as from 0% to less than 15%, from 0% to less than 10%, from 0% to less than 7.5% or from 0% to less than 5%, or such as from 2% to less than 15%, from 2% to less than 10%, from 2% to less than 7.5% or from 2% to less than 5%. In one embodiment the use of a buffer with different pH values between about pH 4 and about pH 8 affects the ionic strength of the buffer by less than 15%, preferably less than 10% or even less than 7.5% or less than 5%, such as from 0% to less than 15%, from 0% to less than 10%, from 0% to less than 7.5% or from 0% to less than 5%, or such as from 2% to less than 15%, from 2% to less than 10%, from 2% to less than 7.5% or from 2% to less than 5%. The use of a multi-component buffer as disclosed herein further allows adjusting the pH of the buffer to the pH of the sample (without changing the buffer composition of the buffer). The use of a multi-component buffer as disclosed herein further allows adjusting the pH of the buffer to near the optimum of the at least one contaminating protein having lipase activity (thereby increasing sensitivity of the method) and/or comparing and identifying conditions that reduce hydrolytic activity.

The reaction solution may further comprise a non-buffering salt. In the present invention any salt that dissociates in water and has no buffering effect may be suitable for adjusting the ionic strength of the reaction solution. Examples for suitable salts are NaCl, KCl, or CaCl₂). In a certain example of the present invention, the non-buffering salt is selected from the group consisting of NaCl, KCl and CaCl₂), preferably the non-buffering salt is NaCl or KCl.

The concentration of the optional non-buffering salt may be in a range of about 100 mM to about 200 mM. In a certain embodiment of the present invention, the non-buffering salt has a concentration of about 100 mM to about 200 mM, preferably about 130 mM to about 170 mM, more preferably about 140 mm to about 150 mM in the reaction mixture. However, ionic strength in the reaction mix should not exceed a certain value due to negative impact on lipase activity. For example, the ionic strength of the optional non-buffering salt is preferably about 200 mM or less, about 190 mM or less, about 180 mM or less, about 170 mM or less, about 160 mM or less, or about 150 mM or less in the reaction mixture, such as from about 100 mM to about 200 mM, preferably about 130 mM to about 170 mM, more preferably about 140 mM to about 150 mM in the reaction mixture. In certain embodiments, the cumulative ionic strength of the ionic strength of the buffer and the non-buffering salt in the reaction mixture does not exceed about 450 mM. Accordingly, the cumulative ionic strength of the buffer and the non-buffering salt the reaction mixture may be about 450 mM or less, about 400 mM or less, about 380 mM or less, about 360 mM or less or about 350 mM or less. For example, the cumulative ionic strength of the buffer and the non-buffering salt the reaction mixture may be about 150 mM to about 450 mM or less, about 150 mM to about 400 mM or less, about 150 mM to about 380 mM or less, about 150 mM to about 360 mM or less or about 150 mM to about 350 mM or less

The method according to the present invention is suitable for detecting the fluorescence in a fluorescence spectrometer or a microplate spectrophotometer (preferably at λ_{Ex} 330-340 nm, λ_{Em} 450 nm). Thus, the reaction mixture is contained (and preferably mixed) in a cuvette or a microtiter plate, preferably at least a 96-well microtiter plate for measurement. The method according to the present invention is therefore particularly suitable for high throughput analysis and/or automated analyses of samples. In certain embodiments, in the method according to the present invention at least 2, 3, 4, 5, 10 or more samples are analyzed simultaneously. Further, each sample is preferably measured at least in triplicates. Preferably, the method according to the invention is therefore performed using a microtiter plate having 96 wells or a multiple of 96 wells. Microtiter plates are not only be used for measuring hydrolysis in step (d), but also for contacting the at least one sample with a reaction solution in step (d) and incubating the sample with the substrate in the reaction mixture in step (c). Thus, in certain embodiment the samples are contacted, incubated and measured in a microtiter plate format having 96 wells or a multiple of 96 wells.

In certain embodiments, the sample is provided at about 30% (v/v) or less, preferably at about 25% (v/v) or less of the reaction mixture. Thus, the sample may be provided at about 20% (v/v) to about 30% (v/v) of the reaction mixture, preferably at about 20% (v/v) to about 25% (v/v) of the reaction mixture. Optionally the sample may be pre-diluted. The at least one sample comprising a recombinant protein may be a harvested cell culture fluid (HCCF) or a cell lysate, an in-process control (IPC) sample, a drug substance sample or a drug product sample, preferably an IPC sample, a drug substance sample or a drug product sample. Preferably, contacting the at least one sample with a reaction solution to form a reaction mixture comprises mixing the at least one sample with the reaction solution to obtain a homogenous reaction mixture. This is preferably done by adding the smaller volume (typically the sample) first and adding the larger volume (typically the reaction solution) second. Preferably, the components of the reaction solution are added as a master mix, wherein the master mix may be prepared as a concentrate that is diluted to working concentration prior to addition to the sample.

Further, the buffer, the non-denaturing surfactant (non-ionic or zwitter-ionic), and the optional non-buffering salt are preferably premixed as an assay buffer that is at least about 3-fold or about 3 to about 5-fold concentrated relative to the reaction mixture. The assay buffer may be stored before use. Alternatively, the assay buffer is provided as a dry mixture. Such dry mixture may be reconstituted with water to provide said at least about 3-fold concentrated or about 3-fold to about 5-fold concentrated assay buffer relative to a final reaction mixture. The substrate is added before use to the assay buffer to provide the reaction solution; preferably, the substrate is added immediately before use to the assay buffer. Thus, the buffer, the surfactant, the substrate and the optional non-buffering salt are preferably premixed as a master mix. The components of the master mix are identical to the components in the reaction solution. The master mix may be prepared as a concentrate that is diluted to working concentration prior to addition to the sample.

In certain embodiment, the buffer, the non-denaturing surfactant (non-ionic or zwitter-ionic), the substrate and the optional non-buffering salt are added as a master mix, wherein the master mix is provided at about 70% (v/v) or more, at about 75% (v/v) or more. Thus, the master mix may be provided at about 70% (v/v) to about 80% (v/v), preferably at about 75% (v/v) to about 80% (v/v).

The at least one sample may be a harvested cell culture fluid (HCCF) or a cell lysate, an in-process control (IPC) sample, a drug substance sample or a drug product sample. The recombinant protein in the sample for detecting lipase activity is preferably a therapeutic protein, such as an antibody, an antibody fragment, an antibody derived molecule, a fusion protein (e.g., an Fc fusion protein), a growth factor, a cytokine or a hormone, preferably an antibody, an antibody fragment, an antibody derived molecule or an Fc fusion protein. Thus, the recombinant protein is preferably a secreted protein. The term “harvested cell culture fluid” or “HCCF” as used herein refers to the cell culture supernatant following harvest, i.e., following separation from the cells. According to the invention the recombinant protein in the sample for detecting lipase activity is not a lipase and/or does not comprise lipase activity. Thus, any lipase activity detected in the at least one sample is contaminating lipase activity and/or derived from at least one contaminating protein having lipase activity, such as host cell proteins (HCPs) derived from the eukaryotic cell. Moreover, the recombinant protein in the sample according to the methods of the present invention is not an esterase or hydrolase and/or does not comprise an esterase or hydrolase activity.

Thus, the method according to the invention can be advantageously used for detecting lipase activity by measuring hydrolysis in a sample comprising an antibody, an antibody fragment, an antibody derived molecule or a fusion protein (e.g., an Fc fusion protein). Typically, an antibody is mono-specific, but an antibody may also be multi-specific. Thus, the method according to the invention may be used for samples comprising mono-specific antibodies, multi-specific antibodies, or fragments thereof, preferably of antibodies (mono-specific), bispecific antibodies, trispecific antibodies or fragments thereof, preferably antigen-binding fragments thereof. Unless specifically mentioned, the term “antibody” refers to a mono-specific antibody. Exemplary antibodies within the scope of the present invention include but are not limited to anti-CD2, anti-CD3, anti-CD20, anti-CD22, anti-CD30, anti-CD33, anti-CD37, anti-CD40, anti-CD44, anti-CD44v6, anti-CD49d, anti-CD52, anti-EGFR1 (HER1), anti-EGFR2 (HER2), anti-GD3, anti-IGF, anti-VEGF, anti-TNFalpha, anti-IL2, anti-IL-5R, anti-IL-36R or anti-IgE antibodies, and are preferably selected from the group consisting of anti-CD20, anti-CD33, anti-CD37, anti-CD40, anti-CD44, anti-CD52, anti-HER2/neu (erbB2), anti-EGFR, anti-IGF, anti-VEGF, anti-TNFalpha, anti-IL2, anti-IL-36R and anti-IgE antibodies. In one embodiment the antibody is an anti-IL-36R antibody, particularly spesolimab. In another embodiment the antibody is not an anti-IL-36R antibody, particularly not spesolimab.

The term “antibody”, “antibodies”, or “immunoglobulin(s)” as used herein relates to proteins selected from among the globulins, which are naturally formed as a reaction of the host organism to a foreign substance (=antigen) from differentiated B-lymphocytes (plasma cells). There are various classes of immunoglobulins: IgA, IgD, IgE, IgG, IgM, IgY, IgW. Preferably the antibody is an IgG antibody, more preferably an IgG1 or an IgG4 antibody. The terms immunoglobulin and antibody are used interchangeably herein. Antibody include monoclonal, monospecific and multi-specific (such as bispecific or trispecific) antibodies, a single chain antibody, an antigen-binding fragment of an antibody (e.g., an Fab or F(ab′)2 fragment), a disulfide-linked Fv, etc. Antibodies can be of any species and include chimeric and humanized antibodies. “Chimeric” antibodies are molecules in which antibody domains or regions are derived from different species. For example, the variable region of heavy and light chain can be derived from rat or mouse antibody and the constant regions from a human antibody. In “humanized” antibodies only minimal sequences are derived from a non-human species. Often only the CDR amino acid residues of a human antibody are replaced with the CDR amino acid residues of a non-human species such as mouse, rat, rabbit or llama. Sometimes a few key framework amino acid residues with impact on antigen binding specificity and affinity are also replaced by non-human amino acid residues. Antibodies may be produced through chemical synthesis, via recombinant or transgenic means, via cell (e.g., hybridoma) culture, or by other means.

Typically antibodies are tetrameric polypeptides composed of two pairs of a heterodimer each formed by a heavy and a light chain. Stabilization of both the heterodimers as well as the tetrameric polypeptide structure occurs via interchain disulfide bridges. Each chain is composed of structural domains called “immunoglobulin domains” or “immunoglobulin regions” whereby the terms “domain” or “region” are used interchangeably. Each domain contains about 70-110 amino acids and forms a compact three-dimensional structure. Both heavy and light chain contain at their N-terminal end a “variable domain” or “variable region” with less conserved sequences which is responsible for antigen recognition and binding. The variable region of the light chain is also referred to as “VL” and the variable region of the heavy chain as “VH”.

Antigen-binding fragments include without being limited thereto e.g. “Fab fragments” (Fragment antigen-binding=Fab). Fab fragments consist of the variable regions of both chains, which are held together by the adjacent constant region. These may be formed by protease digestion, e.g. with papain, from conventional antibodies, but similarly Fab fragments may also be produced by genetic engineering. Further antibody fragments include F(ab′)2 fragments, which may be prepared by proteolytic cleavage with pepsin.

Using genetic engineering methods it is possible to produce shortened antibody fragments which consist only of the variable regions of the heavy (VH) and of the light chain (VL). These are referred to as Fv fragments (Fragment variable=fragment of the variable part). Since these Fv-fragments lack the covalent bonding of the two chains by the cysteines of the constant chains, the Fv fragments are often stabilized. It is advantageous to link the variable regions of the heavy and of the light chain by a short peptide fragment, e.g. of 10 to 30 amino acids, preferably 15 amino acids. In this way a single peptide strand is obtained consisting of VH and VL, linked by a peptide linker. An antibody protein of this kind is known as a single-chain-Fv (scFv). Examples of scFv-antibody proteins are known to the person skilled in the art. Thus, antibody fragments and antigen-binding fragments further include Fv-fragments and particularly scFv.

In recent years, various strategies have been developed for preparing scFv as a multimeric derivative. This is intended to lead, in particular, to recombinant antibodies with improved pharmacokinetic and biodistribution properties as well as with increased binding avidity. In order to achieve multimerisation of the scFv, scFv were prepared as fusion proteins with multimerisation domains. The multimerisation domains may be, e.g. the CH3 region of an IgG or coiled coil structure (helix structures) such as Leucine-zipper domains. However, there are also strategies in which the interaction between the VH/VL regions of the scFv is used for the multimerisation (e.g. dia-, tri- and pentabodies). By diabody the skilled person means a bivalent homodimeric scFv derivative. The shortening of the linker in a scFv molecule to 5-10 amino acids leads to the formation of homodimers in which an inter-chain VH/VL-superimposition takes place. Diabodies may additionally be stabilized by the incorporation of disulphide bridges. Examples of diabody-antibody proteins are known from the prior art.

By minibody the skilled person means a bivalent, homodimeric scFv derivative. It consists of a fusion protein which contains the CH3 region of an immunoglobulin, preferably IgG, most preferably IgG1 as the dimerisation region which is connected to the scFv via a Hinge region (e.g. also from IgG1) and a linker region. Examples of minibody-antibody proteins are known from the prior art.

By triabody the skilled person means a: trivalent homotrimeric scFv derivative. ScFv derivatives wherein VH-VL is fused directly without a linker sequence lead to the formation of trimers.

The skilled person will also be familiar with so-called miniantibodies which have a bi-, tri- or tetravalent structure and are derived from scFv. The multimerisation is carried out by di-, tri- or tetrameric coiled coil structures. In a preferred embodiment of the present invention, the gene of interest is encoded for any of those desired polypeptides mentioned above, preferably for a monoclonal antibody, a derivative or fragment thereof.

The immunoglobulin fragments composed of the CH2 and CH3 domains of the antibody heavy chain are called “Fc fragments”, “Fc region” or “Fc” because of their crystallization propensity (Fc=fragment crystallizable). These may be formed by protease digestion, e.g. with papain or pepsin from conventional antibodies but may also be produced by genetic engineering. The N-terminal part of the Fc fragment might vary depending on how many amino acids of the hinge region are still present.

Antibodies comprising an antigen-binding fragment and an Fc region may also be referred to as full-length antibody. Full-length antibody may be mono-specific and multispecific antibodies, such as bispecific or trispecific antibodies.

Preferred therapeutic antibodies according to the invention are multispecific antibodies, particularly bispecific or trispecific antibodies. Bispecific antibodies typically combine antigen-binding specificities for target cells (e.g., malignant B cells) and effector cells (e.g., T cells, NK cells or macrophages) in one molecule. Exemplary bispecific antibodies, without being limited thereto are diabodies, BiTE (Bi-specific T-cell Engager) formats and DART (Dual-Affinity Re-Targeting) formats. The diabody format separates cognate variable domains of heavy and light chains of the two antigen binding specificities on two separate polypeptide chains, with the two polypeptide chains being associated non-covalently. The DART format is based on the diabody format, but it provides additional stabilization through a C-terminal disulfide bridge. Trispecific antibodies are monoclonal antibodies which combine three antigen-binding specificities. They may be build on bispecific-antibody technology that reconfigures the antigen-recognition domain of two different antibodies into one bispecific molecule. For example, trispecific antibodies have been generated that target CD38 on cancer cells and CD3 and CD28 on T cells. Multispecific antibodies are particularly difficult to product with high product quality.

Another preferred therapeutic protein is a fusion protein, such as an Fc-fusion protein. Thus, the invention can be advantageously used for production of fusion proteins, such as Fc-fusion proteins. Furthermore, the method of increasing protein producing according to the invention can be advantageously used for production of fusion proteins, such as Fc-fusion proteins.

The effector part of the fusion protein can be the complete sequence or any part of the sequence of a natural or modified heterologous protein. The immunoglobulin constant domain sequences may be obtained from any immunoglobulin subtypes, such as IgG1, IgG2, IgG3, IgG4, IgA1 or IgA2 subtypes or classes such as IgA, IgE, IgD or IgM. Preferentially they are derived from human immunoglobulin, more preferred from human IgG and even more preferred from human IgG1 and IgG2. Non-limiting examples of Fc-fusion proteins are MCP1-Fc, ICAM-Fc, EPO-Fc and scFv fragments or the like coupled to the CH2 domain of the heavy chain immunoglobulin constant region comprising the N-linked glycosylation site. Fc-fusion proteins can be constructed by genetic engineering approaches by introducing the CH2 domain of the heavy chain immunoglobulin constant region comprising the N-linked glycosylation site into another expression construct comprising for example other immunoglobulin domains, enzymatically active protein portions, or effector domains. Thus, an Fc-fusion protein according to the present invention comprises also a single chain Fv fragment linked to the CH2 domain of the heavy chain immunoglobulin constant region comprising e.g. the N-linked glycosylation site.

The recombinant protein of the present invention is produced in a eukaryotic cell. Preferably, the eukaryotic cell used for producing the recombinant protein is a yeast cell (e.g., Saccharomyces Klyveromyces) or a mammalian cell (e.g., hamster or human cells). The mammalian cell is preferably a CHO cell, a HEK 293 cell or a derivative thereof. HEK293 cells include without being limited thereto HEK293 cells, HEK293T cells, HEK293F cells, Expi293F cells or derivatives thereof. Commonly used CHO cells for large-scale industrial production are often engineered to improve their characteristics in the production process, or to facilitate selection of recombinant cells. Such engineering includes, but is not limited to increasing apoptosis resistance, reducing autophagy, increasing cell proliferation, altered expression of cell-cycle regulating proteins, chaperone engineering, engineering of the unfolded protein response (UPR), engineering of secretion pathways and metabolic engineering.

Preferably, CHO cells that allow for efficient cell line development processes are metabolically engineered, such as by glutamine synthetase (GS) knockout and/or dihydrofolate reductase (DHFR) knockout to facilitate selection with methionine sulfoximine (MSX) or methotrexate, respectively.

Preferably, the CHO cell used for producing the recombinant protein is a CHO-DG44 cell, a CHO-K1 cell, a CHO-DXB11 cell, a CHO—S cell, a CHO glutamine synthetase (GS)-deficient cell or a derivative of any of these cells.

TABLE 2
Exemplary CHO production cell lines

| Cell line | Order Number |
|---|---|
| CHO | ECACC No. 8505302 |
| CHO wild type | ECACC 00102307 |
| CHO-K1 | ATCC CCL-61 |
| | ECACC 85051005 |
| CHOZN ® | Merck SAFC |
| | GS −/− and DHFR −/− |
| CHO-DUKX (=CHO duk⁻, | ATCC CRL-9096 |
| CHO/dhfr^{-,}, CHO-DXB11) | |
| CHO-DUKX 5A-HS-MYC | ATCC CRL-9010 |
| CHO-DG44 | Urlaub G, et al., 1983. Cell. |
| | 33: 405-412. |
| CHO Pro-5 | ATCC CRL-1781 |
| CHO-S | Life Technologies A1136401; CHO- |
| | S is derived from CHO variant |
| | Tobey et al. 1962 |

Cells are most preferred, when being established, adapted, and completely cultivated under serum free conditions, and optionally in media, which are free of any protein/peptide of animal origin. Commercially available media such as Ham's F12 (Sigma, Deisenhofen, Germany), RPMI-1640 (Sigma), Dulbecco's Modified Eagle's Medium (DMEM; Sigma), Minimal Essential Medium (MEM; Sigma), Iscove's Modified Dulbecco's Medium (IMDM; Sigma), CD-CHO (Invitrogen, Carlsbad, CA), serum-free CHO Medium (Sigma), and protein-free CHO Medium (Sigma) are exemplary appropriate nutrient solutions. Any of the media may be supplemented as necessary with a variety of compounds, non-limiting examples of which are recombinant hormones and/or other recombinant growth factors (such as insulin, transferrin, epidermal growth factor, insulin like growth factor), salts (such as sodium chloride, calcium, magnesium, phosphate), buffers (such as HEPES), nucleosides (such as adenosine, thymidine), glutamine, glucose or other equivalent energy sources, antibiotics and trace elements. Any other necessary supplements may also be included at appropriate concentrations that would be known to those skilled in the art. For the growth and selection of genetically modified cells expressing a selectable gene a suitable selection agent is added to the culture medium.

The recombinant protein of the method of the invention is produced in eukaryotic cells in cell culture. Following expression, the recombinant protein is harvested and further purified. The recombinant protein may be recovered from the culture medium as a secreted protein in the harvested cell culture fluid (HCCF) or from a cell lysate (i.e., the fluid containing the content of a cell lysed by any means, including without being limited thereto enzymatic, chemical, osmotic, mechanical and/or physical disruption of the cell membrane and optionally cell wall) and purified using techniques well known in the art. The samples obtained and/or analyzed at the various steps of purification are also referred to as in-process control (IPC) samples or process intermediates. The harvest typically includes centrifugation and/or filtration, such as to produce a harvested cell culture fluid or cell lysate, preferably harvested cell culture fluid. Thus, the harvested cell culture fluid or the cell lysate may also be referred to as clarified harvested cell culture fluid or clarified cell lysate. It does not contain living cells and cell debris as well as most cell components have been removed. Clarified typically means centrifugation or filtration, preferably filtration. Further process steps may include affinity chromatography, particularly Protein A column chromatography for antibodies or Fc-containing proteins, to separate the product from contaminants. Further process steps may include acid treatment to inactivate viruses, clarifying the product pool by depth filtration, preferably following acid treatment, to remove cell contaminants, such as HCPs and DNA. Further process steps may include in this order or any other order as may be appropriate in the individual case: ion exchange chromatography, particularly anion exchange chromatography to further remove contaminating cell components and/or cation exchange chromatography to remove product related contaminants, such as aggregates. Further, preferably following process steps may include nanofiltration to further remove viruses and ultrafiltration and diafiltration to concentrate the recombinant protein and to exchange buffer, respectively.

Since lipase activity may be associated with host cell protein contaminants, the method according to the present invention may be particularly useful for analyzing process intermediates after (preferably before and after) purification steps that remove HCPs in order to adapt the relevant step to more efficiently remove lipase activity in the process intermediates, such as before and after affinity chromatography, before and after depth filtration in combination with acid treatment and/or before and after anion exchange chromatography. In some embodiments the method comprises obtaining at least one sample after affinity chromatography, and/or after depth filtration in combination with acid treatment (or after acid treatment and/or after depth filtration) and/or after ion exchange chromatography, such as anion exchange chromatography and/or cation exchange chromatography, preferably anion exchange chromatography. In some embodiments the method comprises obtaining at least one sample before and after affinity chromatography and/or before and after depth filtration in combination with acid treatment (or before and after acid treatment and/or before and after depth filtration) and/or before and after ion exchange chromatography, such as anion exchange chromatography and/or cation exchange chromatography, preferably anion exchange chromatography. The person skilled in the art will know that the sample obtained after a certain method step may be the same as the sample obtained before the following method step, such as the sample obtained after affinity chromatography (e.g., Protein A chromatography) may be the same sample as the sample before acid treatment (or before depth filtration in combination with, i.e., following, acid treatment). As explained above, due to the broad buffering range of the buffer, even lipase activity in samples having different pH values can be compared using the method according to the invention. Other samples that may be analyzed using the method according to the invention are drug substance or drug product samples. Drug substance or drug product samples comprise formulation buffer and therefore often contain polysorbate. At very high concentrations polysorbate can inhibit the reaction due to competition with the substrate. However, due to the sensitivity of the assay, at typical concentrations of 0.4 to 0.8 mg/ml polysorbate, lipase activity can also be determined in a drug substance or drug product sample.

In one aspect provided is a method of manufacturing a recombinant protein of interest comprising the steps of detecting lipase activity in a sample comprising the recombinant protein comprising: (a) providing at least one sample comprising the recombinant protein produced in a eukaryotic cell; (b) contacting the at least one sample with a reaction solution to form a reaction mixture, wherein the reaction solution comprises: (i) a buffer having a pH of about pH 4 to about pH 9, (ii) a non-denaturing surfactant not having an ester-bond, wherein the surfactant is a non-ionic or zwitter-ionic surfactant, (iii) a substrate comprising the chromophore 4-methylumbelliferyl (4-MU) in the form of a 4-MU ester, wherein the 4-MU ester is a saturated unbranched-chain fatty acid (C6-C16) 4-MU ester, and (iv) optionally a non-buffering salt; (c) incubating the sample and the substrate in the reaction mixture; (d) detecting lipase activity by measuring hydrolysis of the 4-MU ester and detecting the fluorescence intensity of the released chromophore 4-MU; optionally measuring hydrolysis by detecting the fluorescence intensity of the released chromophore 4-MU over time, while incubating the sample and the substrate in the reaction mixture according to step (c). The person skilled in the art will understand that the method is for detecting contaminating lipase activity and the lipase activity detected in step (d) is contaminating lipase activity in the at least one sample comprising the recombinant protein, more specifically the recombinant protein of interest. The person skilled in the art will understand that the method further comprises (i) cultivating a eukaryotic cell expressing a recombinant protein of interest in cell culture; (ii) harvesting the recombinant protein; (iii) purifying the recombinant protein; and (iv) optionally formulating the recombinant protein into a pharmaceutically acceptable formulation suitable for administration. Thus, provided is a method of manufacturing a recombinant protein of interest comprising the steps of (i) cultivating a eukaryotic cell expressing a recombinant protein of interest; (ii) harvesting the recombinant protein; (iii) purifying the recombinant protein; and (iv) optionally formulating the recombinant protein into a pharmaceutically acceptable formulation suitable for administration; and (v) obtaining at least one sample comprising the recombinant protein in steps (ii), (iii) and/or (iv); wherein the method further comprises detecting (contaminating) lipase activity in a sample comprising the recombinant protein comprising: (a) providing the at least one sample comprising the recombinant protein produced in a eukaryotic cell of step (v); (b) contacting the at least one sample with a reaction solution to form a reaction mixture, wherein the reaction solution comprises: (i) a buffer having a pH of about pH 4 to about pH 9, (ii) a non-denaturing surfactant not having an ester-bond, wherein the surfactant is a non-ionic or zwitter-ionic surfactant, (iii) a substrate comprising the chromophore 4-methylumbelliferyl (4-MU) in the form of a 4-MU ester, wherein the 4-MU ester is a saturated unbranched-chain fatty acid (C6-C16) 4-MU ester, and (iv) optionally a non-buffering salt; (c) incubating the sample and the substrate in the reaction mixture; (d) detecting (contaminating) lipase activity by measuring hydrolysis of the 4-MU ester and detecting the fluorescence intensity of the released chromophore 4-MU; optionally measuring hydrolysis by detecting the fluorescence intensity of the released chromophore 4-MU over time, while incubating the sample and the substrate in the reaction mixture according to step (c). The reaction solution used in the method of the invention is an aqueous reaction solution. Further the lipase activity detected in step (d) is contaminating lipase activity in the at least one sample comprising the recombinant protein, more specifically the recombinant protein of interest. In certain embodiments the recombinant protein of interest is a therapeutic protein, such as an antibody, an antibody fragment, an antibody derived molecule (e.g., scFv, bi- or multi-specific antibodies) or a fusion protein (e.g., an Fc fusion protein). In one embodiment the antibody is an anti-IL-36R antibody, particularly spesolimab. In another embodiment the antibody is not an anti-IL-36R antibody, particularly not spesolimab.

In certain embodiments the method of manufacturing a recombinant protein of interest according to the invention comprises obtaining at least one sample comprising the recombinant protein in a step of harvesting the recombinant protein (in step (ii)), wherein the sample is a harvested cell culture fluid (HCCF) or a cell lysate; in a step of purifying the recombinant protein (in step (iii)), wherein the sample is an in-process control (IPC) sample; and/or in the optional step of formulating the recombinant protein into a pharmaceutically acceptable formulation suitable for administration (in step (iv)), wherein the sample is a drug substance sample or a drug product sample. Preferably, the method of manufacturing a recombinant protein of interest according to the invention comprises obtaining at least one sample comprising the recombinant protein in step (iii), wherein the sample is an in-process control (IPC) sample, such as comprising obtaining at least one sample after affinity chromatography, after depth filtration following acid treatment (or after acid treatment and/or after acid treatment), and/or after ion exchange chromatography, preferably anion exchange chromatography or cation exchange chromatography. More preferably the method comprises obtaining at least one sample before and after affinity chromatography, before and after depth filtration following acid treatment (or before and after acid treatment and/or before and after acid treatment), and/or before and after ion exchange chromatography, preferably anion exchange chromatography or cation exchange chromatography. The step of detecting lipase activity in a sample comprising the recombinant protein is performed according to and as specified in the method for detecting lipase activity as described herein.

### A Kit for Determining Contaminating Lipase Activity by Measuring Hydrolysis in a Sample

Also provided is a kit for determining contaminating lipase activity in a sample comprising a recombinant protein, comprising: (i) a buffer having a pH of about pH 4 to about pH 9, (ii) a non-denaturing surfactant not having an ester-bond, wherein the surfactant is a non-ionic or zwitter-ionic surfactant, (iii) a substrate comprising the chromophore 4-methylumbelliferyl (4-MU) in the form of a 4-MU ester, wherein the substrate is a saturated unbranched-chain fatty acid (C6 to C16) 4-MU ester, and (iv) optionally a non-buffering salt, and/or (iiv) optionally water for dilution. In one embodiment, the kit further comprises an internal standard that serves as positive control and/or allows to calculate relative values compared the internal standard, such as a commercially available lipase, e.g., porcine pancreatic lipase (PPL). The kit may also comprise one or more microtiter plate having 96 wells or a multiple of 96 wells. The kit components may be provided as solutions and/or dry components, either separately or in a pre-mixed form. In the case of the buffer it may be provided as a dry compound providing a buffer having a pH of about pH 4 to about pH 9 upon dilution or reconstitution.

In certain embodiments, the buffer, the surfactant and the optional non-buffering salt are premixed as an assay buffer. Preferably, said assay buffer is at least about 3-fold concentrated or about 3-fold to about 5-fold concentrated relative to a final reaction mixture. Alternatively, the assay buffer is provided as a dry mixture. Such dry mixture may be reconstituted with water to provide said at least about 3-fold concentrated or 5-fold concentrated assay buffer relative to a final reaction mixture. In one embodiment, a dry mixture of the assay buffer is a lyophilized assay buffer. The substrate is provided separately to be added to the assay buffer before use to provide the reaction solution. Alternative the kit may comprise the buffer, the surfactant, the substrate and the optional non-buffering salt premixed as a master mix. The master mix may be adapted to be provided at about 80% (v/v) to about 70% (v/v) of the reaction mixture, preferably at about 80% to about 75% of the reaction mix. The assay buffer and the reaction solution are aqueous solutions.

For the components of the reaction solution the same applies as specified above for the method of the invention. The substrate comprising the chromophore 4-MU is in the form of saturated unbranched-chain fatty acid (C6 to C16) 4-MU ester, wherein the aliphatic chain of the saturated unbranched-chain fatty acid has from C6 to C16 carbon atoms or preferably from C8 to C12 carbon atoms. Thus, the substrate may be 4-methylumbelliferyl octanoate, 4-methylumbelliferyl nonanoate, 4-methylumbelliferyl decanoate (4-MUD), methylumbelliferyl undecanoate or methylumbelliferyl dodecanoate. In certain embodiments the substrate is selected from the group consisting of 4-methylumbelliferyl octanoate, 4-methylumbelliferyl decanoate (4-MUD) and 4-methylumbelliferyl dodecanoate, in a preferred embodiment the substrate is 4-MUD.

The kit may also further comprise an organic solvent for dissolving the substrate, or the substrate is dissolved in an organic solvent. The substrate may be provided as a dry substance and optionally an additional organic solvent or dissolved as a stock solution (such as a 100× stock solution relative to the concentration in the reaction mixture) in an organic solvent, such as dimethyl sulfoxide (DMSO) or dimethyl formamide (DMF), preferably DMSO. Thus, in certain embodiments the substrate is provided as a stock solution of about 100 μM to about 100 mM, preferably about 100 UM to 30 mM, preferably 100 μM to 3 mM, more preferably about 300 μM to 3 μM. In certain embodiments the substrate is provided as stock solution in an organic solvent, wherein the stock solution is added at about 1% to about 5% (v/v) of the reaction mix.

Examples for suitable non-denaturing zwitter-ionic surfactants and not having an ester bond are without being limited thereto 3-[(3-cholamidopropyl)dimethylammonio]-1-propanesulfonate (CHAPS), 3-([3-Cholamidopropyl)dimethylammonio]-2-hydroxy-1-propanesulfonate (CHAPSO), CHAPS analogs (such as Big CHAP N,N-bis-(3-D-gluconamidopropyl)deoxycholamide), Zwittergent (different lengths, such as n-Dodecyl-N,N-dimethyl-3-ammonio-1-propanesulfonate (Zwittergent 3-12)) and 3-[N, N-Dimethyl(3-palmitoylaminopropyl)ammonio]-propanesulfonate or other amidosulfobetaine detergents. Examples for suitable non-denaturing non-ionic surfactants are without being limited thereto pyranoside surfactants (such as Octyl β-D-glucopyranoside (OGP), Nonyl ß-D-glucopyranoside, Dodecyl β-D-maltopyranoside (DDM) or Octyl β-D-thioglucopyranoside), polyoxyethylene (23) lauryl ether (Brij 35) or other Polyoxyethylene ether; saponins (e.g. Digitonin), octylphenoxy polyethoxyethanol (IGEPAL CA-630), poloxamer 188, 338, 407 or tergitol. In certain embodiments, the surfactant is a non-denaturing non-ionic or zwitter-ionic surfactant not having an ester-bond, preferably the surfactant is not polyethylene glycol tert-octylphenyl ether (Triton X-100) and not polyethylene glycol nonylphenyl ether (NP-40). In a preferred embodiment the surfactant is a non-denaturing non-ionic or zwitter-ionic surfactant selected from the group consisting of CHAPS, CHAPSO, Zwittergent (such as Zwittergent 3-12) and a saponin, preferably CHAPS.

In certain embodiments, the buffer comprises one or more buffer substances selected from the group consisting of formic acid, acetic acid, lactic acid, citric acid, malic acid, maleic acid, glycine, glycylglycine, succinic acid, TES (2-{[tris(hydroxymethyl)methyl]amino}ethanesulfonic acid), MOPS (3-(N-morpholino)propanesulfonic acid), PIPES (piperazine-N,N′-bis(2-ethanesulfonic acid)), MES (2-(N-morpholino)ethanesulfonic acid), Tris base, Tris, Bis-Tris, Bis-Tris-Propane, Bicine (N,N-bis(2-hydroxyethyl)glycine), HEPES (4-2-hydroxyethyl-1-piperazineethanesulfonic acid), TAPS (3-([tris(hydroxymethyl)methyl]amino}pro-panesulfonic acid), Tricine (N-tris(hydroxymethyl)methylglycine), Na₂HPO₄ and NaH₂PO₄. In certain embodiments the buffer has a pH of about pH 4 to about pH 8, preferably the buffer has a pH of about pH 5 to about pH 7.5, more preferably the buffer has a pH of about pH 5.5 to about pH 7.5.

The buffer may comprise a single buffer substance or may be a multiple component buffer as specified above for the method according to the invention. The multi-component buffer may comprise more than one buffer substance with overlapping buffering ranges in order to have a broader buffering range. The buffer may, e.g., comprise two, three, four, five or more buffering substances, preferably two or more buffering substances, more preferably three or more buffering substances. For example the multi-component buffer may comprise two to four buffering substances, three to four buffering substances, more preferably 3 buffering substances. In certain embodiments, the multi-component buffer comprises at least three buffer substances with overlapping buffering ranges, preferably comprising at least one of Tris, MES and/or acetic acid, more preferably acetic acid, MES and Tris at a ratio of 1:1:2. In certain embodiments the buffer is a multi-component buffer having a buffering range from at least about pH 5 to at least about pH 7.5, preferably from at least about pH 4 to at least about pH 8. In one embodiment, the use of the multi-component buffer at different pH values between about pH 4 and about pH 9 affects the ionic strength of the buffer by less than 15%, preferably less than 10% or even less than 7.5% or less than 5%, such as from 0% to less than 15%, from 0% to less than 10%, from 0% to less than 7.5% or from 0% to less than 5%, or such as from 2% to less than 15%, from 2% to less than 10%, from 2% to less than 7.5% or from 2% to less than 5%. The optional non-buffering salt may be, e.g., NaCl, KCl and CaCl₂) and is preferably NaCl or KCl.

In view of the above, it will be appreciated that the invention also encompasses the following items:
Item 1 provides a method for detecting lipase activity in a sample comprising a recombinant protein comprising (a) providing at least one sample comprising a recombinant protein produced in a eukaryotic cell; (b) contacting the at least one sample with a reaction solution to form a reaction mixture, wherein the reaction solution comprises: (i) a buffer having a pH of about pH 4 to about pH 9, (ii) a non-denaturing surfactant not having an ester-bond, wherein the surfactant is a non-ionic or zwitter-ionic surfactant, (iii) a substrate comprising the chromophore 4-methylumbelliferyl (4-MU) in the form of a 4-MU ester, wherein the 4-MU ester is a saturated unbranched-chain fatty acid (C6-C16) 4-MU ester, and (iv) optionally a non-buffering salt; (c) incubating the sample and the substrate in the reaction mixture; and (d) detecting lipase activity by measuring hydrolysis of the 4-MU ester and detecting the fluorescence intensity of the released chromophore 4-MU; optionally measuring hydrolysis by detecting the fluorescence intensity of the released chromophore 4-MU over time, while incubating the sample and the substrate in the reaction mixture according to step (c).
Item 2 specifies the method of item 1 or 2, wherein the fluorescence intensity of the released chromophore 4-MU is detected without stopping hydrolysis of the 4-MU ester; and/or the sample and the substrate in the reaction mixture are incubated for any time period between 2 min and less than 5 hours, less than 3 hours, less than 2 hours, or less than 0.5 hours.
Item 3 specifies the method of any one of the preceding item, wherein the fluorescence intensity of the released chromophore 4-MU is detected over time and follows a pseudo-zero order reaction rate, and optionally wherein a reaction mixture that does not meet the requirement of pseudo-zero order reaction rate is excluded from analysis.
Item 4 specifies the method of any one of the preceding items, further comprising a step of (a) determining the rate of hydrolysis by detecting the fluorescence intensity of the released chromophore 4-MU as relative fluorescent units (RFU) and determining the amount of the released chromophore 4-MU (mol/s) by comparing it to a calibration curve generated by using defined concentrations of 4-MU, and/or (b) calculating a relative value compared to an internal standard.
Item 5 specifies the method of any one of the preceding items, wherein the lipase activity detected in the at least one sample is contaminating lipase activity.
Item 6 specifies the method of any one of the preceding items, wherein the substrate is selected from the group consisting of 4-methylumbelliferyl octanoate, 4-methylumbelliferyl nonanoate, 4-methylumbelliferyl decanoate (4-MUD), 4-methylumbelliferyl undecanoate and 4-methylumbelliferyl dodecanoate.
Item 7 specifies the method of any one of the preceding items, wherein the substrate is provided at a final concentration of about 1 μM to about 1 mM in the reaction mixture.
Item 8 specifies the method of any one of the preceding items, wherein the substrate is provided as stock solution in an organic solvent, and wherein the stock solution is added at about 1% to about 5% (v/v) of the reaction mix and/or wherein the organic solvent is DMSO or DMF.
Item 9 specifies the method of any one of the preceding items, wherein the surfactant has a final concentration in the reaction mixture above its critical micelle concentration in the reaction mixture. Item 10 specifies the method of any one of the preceding items, wherein the surfactant is selected from the group consisting of CHAPS, CHAPSO and Zwittergent, preferably CHAPS, and/or and is not polyethylene glycol tert-octylphenyl ether (Triton X-100) and not polyethylene glycol nonylphenyl ether (NP-40).
Item 11 specifies the method of any one of the preceding items, wherein the surfactant is CHAPS and is provided at a final concentration in the reaction mixture of about 8 mM to about 20 mM, preferably at about 8 mM to about 15 mM, more preferably at about 10 mM.
Item 12 specifies the method of any one of the preceding items, wherein the buffer comprises one or more buffer substances selected from the group consisting of a formic acid, acetic acid, lactic acid, citric acid, malic acid, maleic acid, glycine, glycylglycine, succinic acid, TES (2-{[tris(hydroxymethyl) methyl]amino}ethanesulfonic acid), MOPS (3-(N-morpholino)propanesulfonic acid), PIPES (piperazine-N,N′-bis(2-ethanesulfonic acid)), MES (2-(N-morpholino)ethanesulfonic acid), Tris base, Tris, Bis-Tris, Bis-Tris-Propane, Bicine (N,N-bis(2-hydroxyethyl)glycine), HEPES (4-2-hydroxyethyl-1-piperazineethanesulfonic acid), TAPS (3-([tris(hydroxymethyl)methyl]amino}pro-panesulfonic acid), Tricine (N-tris(hydroxymethyl)methylglycine), Na₂HPO₄ and NaH₂PO₄.
Item 13 specifies the method of any one of the preceding items, wherein the buffer has a pH of about 5 to about 7.5, preferably the buffer has a pH of about 5.5 to about 7.5.
Item 14 specifies the method of any one of the preceding items, wherein the buffer is a multi-component buffer having a buffering range from at least about pH 5 to at least about pH 7.5, preferably from at least about pH 4 to at least about pH 8.
Item 15 specifies the method of item 14, wherein the multi-component buffer comprises at least three buffer substances with overlapping buffering ranges, preferably comprising at least one of Tris, MES and/or acetic acid.
Item 16 specifies the method of item 14 or 15, wherein the method comprises (a) the use of a buffer with a variable pH from at least about pH 4 to at least about pH 8; (b) the use of a buffer with different pH values between about pH 4 and about pH 8 thereby affecting the ionic strength by less than 15%, preferably less than 10%, preferably from 0% to less than 15%, from 0% to less than 10%, from 0% to less than 7.5% or from 0% to less than 5%; (c) adjusting the pH of the buffer to the pH of the sample; (d) adjusting the pH of the buffer to near the optimum of the at least one (contaminating) protein having lipase activity; or (e) comparing and identifying conditions that reduce hydrolytic activity.
Item 17 specifies the method of any one of the preceding items, wherein at least 2, 3, 4, 5, 10 or more samples are analyzed simultaneously.
Item 18 specifies the method of any one of the preceding items, wherein the samples are contacted, incubated and measured in a plate format having 96 wells or a multiple of 96 wells.
Item 19 specifies the method of any one of the preceding items, wherein the sample is provided at about 20% to about 30% (v/v) of the reaction mixture, preferably at about 25% of the reaction mixture, optionally wherein the sample may be pre-diluted.
Item 20 specifies the method of any one of the preceding items, wherein the buffer, the surfactant and the optional non-buffering salt are premixed as an assay buffer that is about 3 to about 5-fold concentrated relative to the reaction mixture.
Item 21 specifies the method of any one of the preceding items, wherein the buffer, the surfactant, the substrate and the optional non-buffering salt are added as a master mix to the sample, wherein the master mix is provided at about 80% (v/v) to about 70% (v/v) of the reaction mixture, preferably at about 75% of the reaction mix.
Item 22 specifies the method of any one of the preceding items, wherein the non-buffering salt is selected from the group consisting of NaCl, KCl and CaCl₂), preferably wherein the non-buffering salt is NaCl or KCl.
Item 23 specifies the method of any one of the preceding items, wherein the non-buffering salt has a concentration of about 100 mM to about 200 mM, preferably about 130 mM to about 170 mM, more preferably about 140 mM to about 150 mM in the reaction mixture.
Item 24 specifies the method of any one of the preceding items, wherein the ionic strength of non-buffering salt is about 200 mM or less in the reaction mixture, preferably about 150 mM or less in the reaction mixture, preferably about 100 mM to about 200 mM, preferably about 130 mM to about 170 mM, more preferably about 140 mM to about 150 mM in the reaction mixture.
Item 25 specifies the method of any one of the preceding items, wherein the cumulative ionic strength of the buffer and the non-buffering salt in the reaction mixture is about 450 mM or less, preferably about 400 mM, more preferably 350 mM or less in the reaction mixture.
Item 26 specifies the method of any one of the preceding items, wherein the fluorescence is detected using a fluorescence spectrometer or microplate spectrophotometer.
Item 27 specifies the method of any one of the preceding items, wherein the at least one sample is a harvested cell culture fluid (HCCF) or a cell lysate, an in-process control (IPC) sample, a drug substance sample or a drug product sample.
Item 28 specifies the method of any one of the preceding items, wherein (a) the recombinant protein is not a lipase and/or an enzyme having lipase activity; and/or (b) the recombinant protein is selected from the group consisting of an antibody, an antibody fragment, an antibody derived molecule and a fusion protein.
Item 29 specifies the method of any one of the preceding items, wherein the eukaryotic cell used for producing the recombinant protein is a yeast cell or a mammalian cell, wherein the mammalian cell is preferably a CHO cell, a HEK 293 cell or a derivative thereof.
Item 30 provides a kit for determining contaminating lipase activity in a sample comprising a recombinant protein comprising: (i) a buffer having a pH of about pH 4 to about pH 9; (ii) a non-denaturing surfactant not having an ester-bond, wherein the surfactant is a non-ionic or zwitter-ionic surfactant; and (iii) a substrate comprising the chromophore 4-methylumbelliferyl (4-MU) in the form of a 4-MU ester, wherein the substrate is a saturated unbranched-chain fatty acid (C6 to C16) 4-MU ester; and (iv) optionally a non-buffering salt; and/or (v) optionally water for dilution.
Item 31 specifies the kit of item 30, wherein the substrate is selected from the group consisting of 4-methylumbelliferyl octanoate, 4-methylumbelliferyl nonanoate, 4-methylumbelliferyl decanoate (4-MUD), 4-methylumbelliferyl undecanoate and 4-methylumbelliferyl dodecanoate.
Item 32 specifies the kit of item 30 or 31, wherein the kit further comprises an organic solvent for dissolving the substrate, preferably DMSO or DMF.
Item 33 specifies the kit of any one of items 30 to 32, wherein the surfactant is not polyethylene glycol tert-octylphenyl ether (Triton X-100) and not polyethylene glycol nonylphenyl ether (NP-40) or wherein the surfactant is a non-denaturing zwitter-ionic surfactant selected from the group consisting of CHAPS, CHAPSO and Zwittergent, preferably CHAPS.
Item 34 specifies the kit of any one of items 30 to 33, wherein the buffer comprises one or more buffer substances selected from the group consisting of a formic acid, acetic acid, lactic acid, citric acid, malic acid, maleic acid, glycine, glycylglycine, succinic acid, TES (2-{[tris(hydroxymethyl)methyl]amino}ethanesulfonic acid), MOPS (3-(N-morpholino)propanesulfonic acid), PIPES (piperazine-N,N′-bis(2-ethanesulfonic acid)), MES (2-(N-morpholino)ethanesulfonic acid), Tris base, Tris, Bis-Tris, Bis-Tris-Propane, Bicine (N, N-bis(2-hydroxyethyl)glycine), HEPES (4-2-hydroxyethyl-1-piperazineethanesulfonic acid), TAPS (3-([tris(hydroxymethyl)methyl]amino}pro-panesulfonic acid), Tricine (N-tris(hydroxymethyl)methylglycine), Na₂HPO₄ and NaH₂PO₄.
Item 35 specifies the kit of any one of items 30 to 34, wherein the buffer has a pH of about 5 to about 7.5, preferably the buffer has a pH of about 5.5 to about 7.5.
Item 36 specifies the kit of any one of items 30 to 35, wherein the buffer is a multi-component buffer having a buffering range from at least about pH 5 to at least about pH 7.5, preferably from at least about pH 4 to at least about pH 8.
Item 37 specifies the kit of item 36, wherein the multi-component buffer comprises at least three buffer substances with overlapping buffering ranges, preferably comprising at least one of Tris, MES and/or acetic acid.
Item 38 specifies the kit of any one of items 30 to 37, wherein the kit further comprises one or more microtiter plate having 96 wells or a multiple of 96 wells.
Item 39 specifies the kit of any one of items 30 to 38, wherein the buffer, the surfactant and the optional non-buffering salt are premixed as an assay buffer that is at least about 3-fold or about 3 to about 5-fold concentrated relative to a final reaction mixture and/or provided as a dry mixture.
Item 40 specifies the kit of any one of items 30 to 39, wherein the non-buffering salt is selected from the group consisting of NaCl, KCl and CaCl₂), preferably wherein the non-buffering salt is NaCl or KCl.
Item 41 provides a method of manufacturing a recombinant protein of interest comprising the steps of (i) cultivating a eukaryotic cell expressing a recombinant protein of interest in cell culture; (ii) harvesting the recombinant protein; (iii) purifying the recombinant protein; and (iv) optionally formulating the recombinant protein into a pharmaceutically acceptable formulation suitable for administration; and (v) obtaining at least one sample comprising the recombinant protein in steps (ii), (iii) and/or (iv); wherein the method further comprises detecting (contaminating) lipase activity in a sample comprising the recombinant protein comprising: (a) providing the at least one sample comprising the recombinant protein produced in a eukaryotic cell of step (v); (b) contacting the at least one sample with a reaction solution to form a reaction mixture, wherein the reaction solution comprises: (i) a buffer having a pH of about pH 4 to about pH 9, (ii) a non-denaturing surfactant not having an ester-bond, wherein the surfactant is a non-ionic or zwitter-ionic surfactant, (iii) a substrate comprising the chromophore 4-methylumbelliferyl (4-MU) in the form of a 4-MU ester, wherein the 4-MU ester is a saturated unbranched-chain fatty acid (C6-C16) 4-MU ester, and (iv) optionally a non-buffering salt; (c) incubating the sample and the substrate in the reaction mixture; (d) detecting (contaminating) lipase activity by measuring hydrolysis of the 4-MU ester and detecting the fluorescence intensity of the released chromophore 4-MU; optionally measuring hydrolysis by detecting the fluorescence intensity of the released chromophore 4-MU over time, while incubating the sample and the substrate in the reaction mixture according to step (c). In preferred embodiments, the surfactant in step (b) (ii) is not polyethylene glycol tert-octylphenyl ether (Triton X-100) and not polyethylene glycol nonylphenyl ether (NP-40). Preferably, the surfactant is a non-denaturing zwitter-ionic surfactant, such as selected from the group consisting of CHAPS, CHAPSO and Zwittergent, preferably CHAPS.
Item 42 specifies the method of manufacturing a recombinant protein of interest according to item 41 wherein the method comprises obtaining at least one sample comprising the recombinant protein in in step (ii), wherein the sample is a harvested cell culture fluid (HCCF) or a cell lysate; in step (iii), wherein the sample is an in-process control (IPC) sample; and/or in step (iv), wherein the sample is a drug substance sample or a drug product sample.
Item 43 specifies the method of manufacturing a recombinant protein of interest according to item 41 or 42 to comprise obtaining at least one sample comprising the recombinant protein in step (iii), wherein the sample is an in-process control (IPC) sample.
Item 44 specifies the method of manufacturing a recombinant protein of interest according to item 43, wherein the method comprises obtaining at least one sample after affinity chromatography, after depth filtration following acid treatment (or after acid treatment and/or after depth filtration), and/or after anion exchange chromatography, preferably obtaining at least one sample before and after affinity chromatography, before and after depth filtration following acid treatment (or before and after acid treatment and/or before and after depth filtration), and/or before and after anion exchange chromatography.
Item 45 specifies the method of manufacturing a recombinant protein of interest according to any one of items 41 to 44, comprising detecting lipase activity in a sample comprising the recombinant protein according to the method of any one of items 1-29.

### EXAMPLES

### Selection of 4-MU as a Chromophore

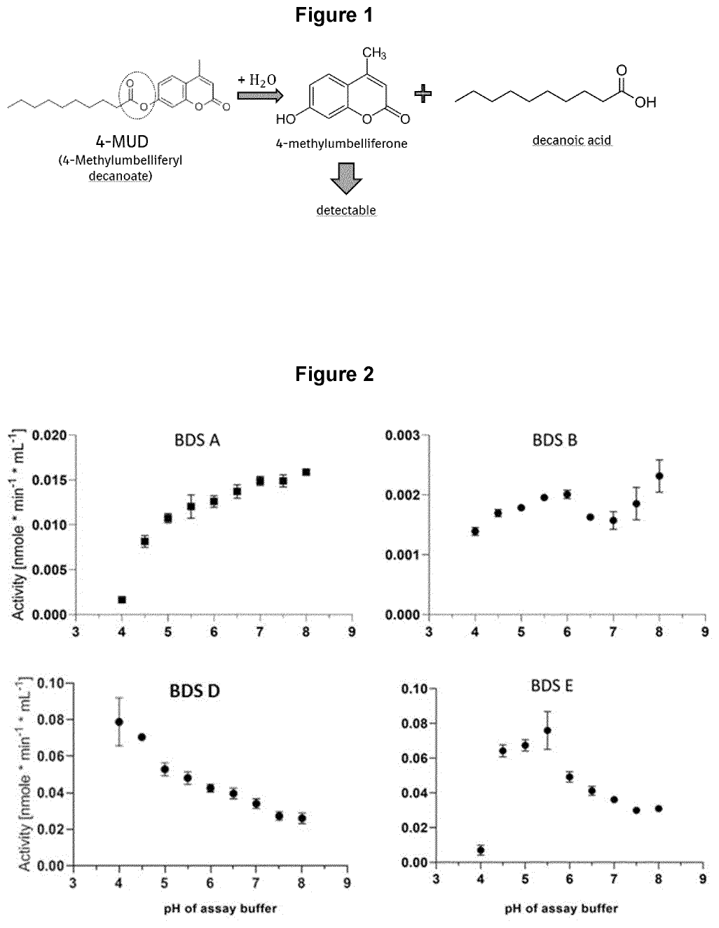

Many lipases are able to hydrolyze fatty acid esters of 4-Methylumbelliferone (4-MU). Upon hydrolysis not only the fatty acid is released but also the highly fluorescent 4-MU (FIG. 1). The increase of fluorescence is directly proportional to the rate of hydrolysis and can therefore be used to determine the hydrolytic activity or lipase activity in a given sample.

4-Methylumbelliferyl was chosen as detection agent because its spectral characteristics combine a high quantum yield with a sufficiently insensitivity to altering ionic strength and pH (data not shown). These characteristics support a robust assay performance. Further, it unlocks the highly sensitive detection principle based on fluorescence that is sufficiently insensitive to disturbances, caused by e.g. light scattering (data not shown).

### Lipase Assay

For the lipase assay two different buffers have been used. A phosphate assay buffer or a multi-component buffer, the AMT buffer. The phosphate buffer comprises 108 mM Na₂HPO₄, 25 mM NaH₂PO₄, 186.2 mM NaCl, 13.3 mM CHAPS, at pH 7.4 resulting in a final concentration in the reaction mixture of 81 mM Na₂HPO₄, 19 mM NaH₂PO₄, 140 mM NaCl, 10 mM CHAPS, pH 7.4. The AMT assay buffer with a broad buffering range of 4-8 is provided as a 4× stock solution and comprises 0.3 M acetic acid, 0.3 M MES, 0.6 M Tris, 0.6 M NaCl, 40 mM CHAPS, with the pH adjusted as indicated using HCl or NaOH (recommended pH range: 4-8), resulting in a final concentration in the reaction mixture of 75 mM acetate, 75 mM MES, 150 mM Tris, 150 mM NaCl, and 10 mM CHAPS.

The substrate was stored at a concentrated stock solution comprising 3 mM 4-methylumbelliferyl decanoate (4-MUD; FM25973, Carbosynth) in DMSO and diluted 1:10 in DMSO prior to use resulting in a 100× stock solution for use comprising 0.3 mM in DMSO.

In order to ensure comparability of the measurements, the assay buffer and the substrate have been mixed prior to use. The mastermix has been prepared immediately before use and the reaction was started by mixing the given sample (e.g. drug substance) with the mastermix including the substrate and the assay buffer and optionally additional water. Mixing in the reaction vessel has been performed by providing the smaller volume to the reaction vessel prior to adding the larger volume of the two components, sample and mastermix. Thus, typically the sample has been added first.

The reaction mixtures have been prepared as follows. For the assay based on the phosphate buffer (const. pH 7.4), A) cuvette: mastermix (2250 μL phosphate assay buffer, 30 μL 0.3 mM 4-MUD in DMSO) has been added to 720 μL sample in the reaction vessel, B) per well in a 96 well plate: mastermix (225 μL phosphate assay buffer, 3 μL 0.3 mM 4-MUD in DMSO) has been added to 72 μL sample in the reaction vessel. For the assay based on AMT buffer (also referred to as 3-component buffer (variable pH), C) cuvette: mastermix (750 μL 4×AMT assay buffer, 1500 μL H₂O, 30 μL 0.3 mM 4-MUD in DMSO) has been added to 720 μL sample in the reaction vessel, D) per well in a 96 well plate: mastermix (75 μL 4×AMT assay buffer, 150 μL H₂O, 3 μL 0.3 mM 4-MUD in DMSO) has been added to 72 μL sample in the reaction vessel.

Hydrolysis of the substrate 4-MUD has been measured by detecting the fluorescence intensity of the released chromophore 4-MU immediately following mixing in real-time for a few minutes up to 5 hours depending on fluorescence intensity. Fluorescence (λₑₘ=450 nm, λₑₓ=330 or 340 nm) indicating 4-MU release has been monitored either in a cuvette using a fluorescence spectrometer or in a 96-well plate using a suitable microplate reader at 25° C. High correlation was found between the use of a fluorescence spectrometer (Fluoromax4, Horiba Jobin Yvon) and a plate reader SpectraMax M3 (Molecular Devices) (see FIG. 9). Negative controls comprising no samples were included to exclude potential auto-hydrolysis. A linear fit has been used to calculate the slope (e.g. RFU/s). Detecting fluorescence in real-time allows measurement in a time-frame with a pseudo-zero order reaction rate. Samples were run at least in triplicates and individual reaction mixtures were excluded from analysis in case they did not meet a pseudo-zero order reaction rate, e.g. due to bubbles in the well etc. It was found that this way of eliminating outliers strongly increases sensitivity of the assay. Calibration curves using defined concentrations of 4-MU can be used to calculate the rate of hydrolysis (e.g. nmol/s). Calibration curves with known 4-MU concentrations further allowed the determination and comparison of reaction velocities at different pH values.

### AMT Buffer Development

The lipase assay has initially been set up using the phosphate assay buffer. For kinetic measurements of hydrolytic activity in different product samples at varying pH the three-component AMT buffer has been developed. The AMT buffer comprising acetic acid, MES and Tris as buffer substances, allows for a wider pH buffering range and hence measurements at a larger pH range or even at different pH. The buffer substances used are known to be non-fluorescent, poor metal chelators and interference with enzymatic activity is unlikely. Comparable to the phosphate assay buffer, CHAPS was added above CMC and ionic strength was adjusted using NaCl. Since the assay turned out to be sensitive to ionic strength it was important to generate a buffer not only comprising buffer substances with overlapping buffer ranges, but also a buffer that only moderately changes (less than 15% preferably even less than 10%) ionic strength at different pH (range pH 4-8) (Ellis K J, Morrisson J F, 1982. Methods in Enzymology, 87: 405-426). The AMT buffer allows, e.g., to identify conditions, including pH conditions that reduce hydrolytic activity. This buffer further allows taking measurements at the pH of the sample to determine lipase activity at the specific conditions present in a sample as well as to compare lipase activity at different states during purification. The assay allows to further increase sensitivity by measuring the sample at pH optimum.

### HPLC-CAD Method

HPLC-CAD was used to quantify the polysorbate content in aqueous solutions. Using an aqueous mobile phase containing isopropanol or equivalent, intact polysorbate was bound to a mixed-mode column, based on a mixture of reversed phase and ion exchange polymers. Polysorbate was then eluted using a mobile phase with acetonitrile or equivalent. More specifically HPLC chromatography was conducted using a mobile phase A (MPA) of 10 mM ammonium formate, pH 4.5, 20% (v/v) 2-propanol and a mobile phase B (MPB) containing 50% (v/v) acetonitrile and 50% 2-propanol (v/v). Separation of protein and matrix components as well as polysorbate degradants was achieved on a mixed mode column (Oasis® Max Online column, 2.1 mm×20 mm, 30 μm, 80 Å) using a flow of 1.0 mL/min in MPA, and intact polysorbate species were eluted by a step-gradient using MPB. The analyte was detected using a charged aerosol detector (CAD). CAD detection employs an inert gas flow system which nebulizes the analyte, removes the mobile phase, and induces the formation of charged particles. The induced current measured is proportional to the quantity of polysorbate contained in the sample. Polysorbate was quantified using an external calibration standard series.

### Fluorescence Micelle Assay

Fluorescent molecules such as N-phenyl-1-naphtylamine (NPN) can be solubilized in the presence of a surfactant in aqueous solutions. Once the surfactant exceeds its critical micelle concentration, a large increase in the fluorescence quantum yield is observed as the fluorescent reagent intercalates into the hydrophobic interior of the micelles. The amount of solubilized fluorescence reagent is directly proportional to the concentration of micelles in the solution. In more detail, a sample was spiked with 5×10⁻⁶ M NPN and fluorescence was detected using a fluorescence detector (λₑₘ=420 nm, λₑₓ=350 nm). Polysorbate was quantified using an external calibration standard series.

### Example 1: Lipase Assay Allows to Measure Activity in Different Drug Substances

The lipase assay has been developed to determine lipase activity in various drug substances following purification and to aid to adapt and improve purification steps during down-stream processing in order to remove lipase activity in the final drug substance responsible for polysorbate degradation in final drug products. The contaminating lipase activity co-purified as host cell proteins present in some drug substance may differ depending on the protein as well as the purification process. Different lipases exhibit specific pH optima, which usually relate to their cellular localization, e.g., lysosomal lipases typically have an acidic pH optimum.

The lipase assay was therefore used for kinetic measurements of hydrolytic activities in different bulk drug substance (BDS) at varying pH. For this the AMT buffer has been established to determine pH dependency within the pH range of 4-8. The BDS was used in undiluted form at 72 μL per well for each sample. Calibration curves with known 4-MU concentrations allowed determining the reaction velocity at each pH in nmol/min/mL. As negative controls blank runs were performed using formulation buffer only to monitor non-enzymatic hydrolysis. Optionally positive controls have been included using a commercially available lipase such as porcine pancreatic lipase (PPL) at ≤0.24 mg/mL. The drug products, referred to as BDS of Product A, B, D and E (BDS A, B, D and E), differed in the amount of hydrolytic activity detected (see FIG. 2) as well as in their hydrolytic activity pH profile. For example, BDS A showed a clear pH optimum at alkaline pH, while BDS D and BDS E rather showed a pH optimum at acidic pH (FIG. 2). At a pH≥7.5 autohydrolysis may account for residual hydrolytic activity (see BDS B and BDS E). This can be verified by detecting residual hydrolytic activity in the presence of a lipase inhibitor such as Orlistat (3.3 μM) or by running a blank sample comprising no lipase activity (sample buffer or medium) in parallel (data not shown). This experiment also demonstrated, that extremely low lipase activity, such as in BDS B were still detectable at pH optimum (pH 6).

Overall it has been found that the pH dependencies differed between the various drug substance samples tested, indicating that different drug substances comprise different contaminating proteins with lipase activity, which are sometimes associated with a distinct pH optimum. This demonstrates that the assay is useful for detecting such divergent enzymes with lipase activity in tested samples.

Additionally different formulation buffers have been tested at different pH demonstrating that different formulation buffers may support or suppress lipase activity in the drug product (data not shown).

### Example 2: Influence of Substrate Concentration

It is important to have enough substrate in solution to achieve a sufficiently high sensitivity of the observed reaction. This need competes with the limited solubility of 4-MUD and its derivatives in the water-based master- and reaction-mix. When added in water 4-MUD at high concentrations instantly formed visible particles. The addition of a surfactant to the assay-mix, e.g. CHAPS, greatly increased solubility of 4-MUD and hence prevented precipitation.

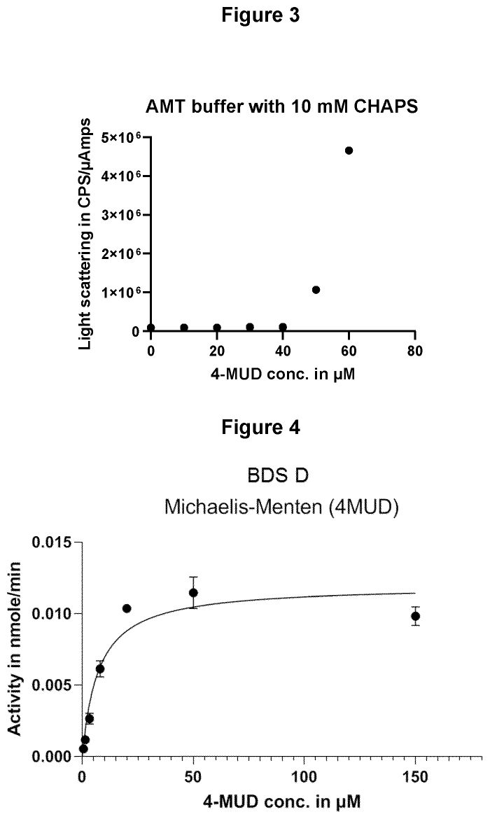

Consequently, the ideal substrate concentration for the intended use was determined by carrying out a set of light scattering and activity experiments. Solubility of 4-MUD was tested using right angle light scattering (RALS) experiments in AMT assay buffer (75 mM acetate, 75 mM MES, 150 mM Tris, 150 mM NaCl, 10 mM CHAPS, pH 7) using a fluorescent spectrometer (λₑₘ=400 nm, λₑₓ=400 nm) in a 1 cm macro-cuvette. It was found that the maximum solubility in the ATM buffer comprising 10 mM CHAPS was about ˜40 UM of 4-MUD (FIG. 3).

Michaelis-Menten kinetics of the hydrolytic activity in a bulk drug substance (BDS D) was analysed (72 μL/well, undiluted BDS D). Assay was performed in AMT assay buffer (75 mM acetate, 75 mM MES, 150 mM Tris, 150 mM NaCl, 10 mM CHAPS, pH 5.5) with varying concentrations of 4-MUD (1.5625 μM-150 UM) using a microplate reader (λₑₘ=450 nm, λₑₓ=330 nm, top read mode) in a black 96 well plate. The pH was adjusted to the pH of the bulk drug substance. As shown in FIG. 4 the speed of the reaction was found to be sufficient with 3 μM of 4-MUD or even less to support a fast read-out. Typically, 4-MU release is measured immediately after mixing and is detected for a few minutes to a few hours, typically for about 20 min to about 2 hours.

As a result, a concentration between 3 μM to 30 μM 4-MUD has proven to be a reasonable compromise to be used as standard concentration (FIG. 4), although concentrations of 1 μM to 1000 UM can be used.

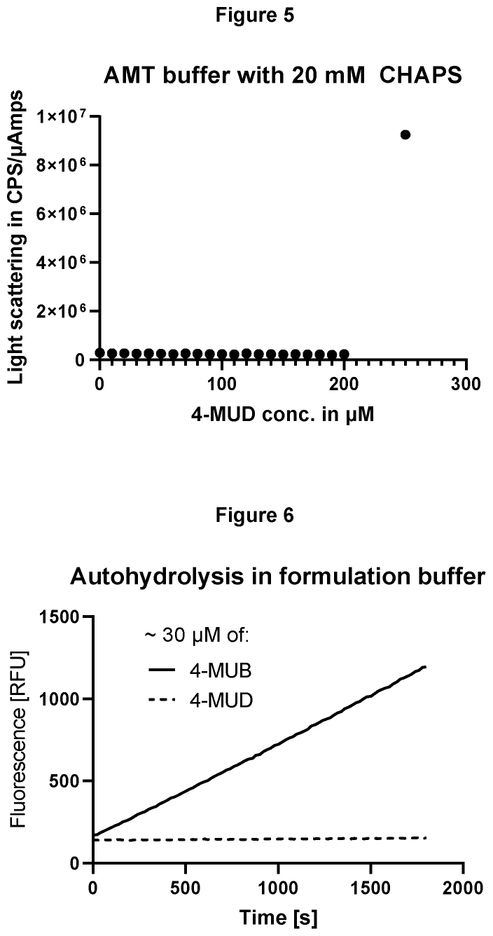

In case of special unit operations and/or troubleshooting activities, higher concentrations may be favorable, e.g. to determine Michaelis-Menten kinetics (FIG. 4). Therefore, either CHAPS concentration may be increased as shown in FIG. 5 or other suitable surfactants can be used (e.g. Zwittergent).

### Example 3: Influence of the Fatty Acid Chain Length

The acyl ester derivate 4-Methyumbelliferyldecanoate (4-MUD) was chosen because decanoate acyl ester capture a broader enzyme spectrum compared to e.g. using oleate, comprising a longer and unsaturated acyl chain. Further, the shorter chain length of decanoate offered better solubility in water-based reaction mixtures compared to e.g. oleate. Consequently, more substrate can be used in the assay mix. More specifically, it was found that solubility becomes strongly limiting at a chain length of C16 or longer (data not shown). Additionally, it was found that the decanoate offers a better resistance to auto-hydrolysis compared to e.g. butyrate (FIG. 6). Auto-hydrolysis of 4-Methyumbelliferylbutyrate (4-MUB) and 4-MUD has been analysed. Fluorescence was monitored for 1800 seconds with 30 μM of 4-MUB and 4-MUD and compared in AMT assay buffer (75 mM acetate, 75 mM MES, 150 mM Tris, 150 mM NaCl, 10 mM CHAPS, pH 5.5) using a microplate reader (λₑₘ=450 nm, λₑₓ=330 nm, top read mode) in a black 96 well plate. We further determined that a chain length up to C5 strongly increased auto-hydrolysis (data not shown). Using a substrate with a C8 chain, but a different chromophore showed more, but still acceptable auto-hydrolysis (data not shown). Taken together, it has been demonstrated that the specific substrate used is important and strongly improves and increases the sensitivity of the assay. The C10 fatty acid in 4-MUD used in the assay was found to be an optimal choice.

### Example 4: Influence of a Surfactant in the Reaction Mix

The reaction conditions should be designed to maintain and support activity of relevant enzymes such as the hydrolytic activity of lipases. Among other things, this requirement was achieved by modifying the reaction mixture of the assay.

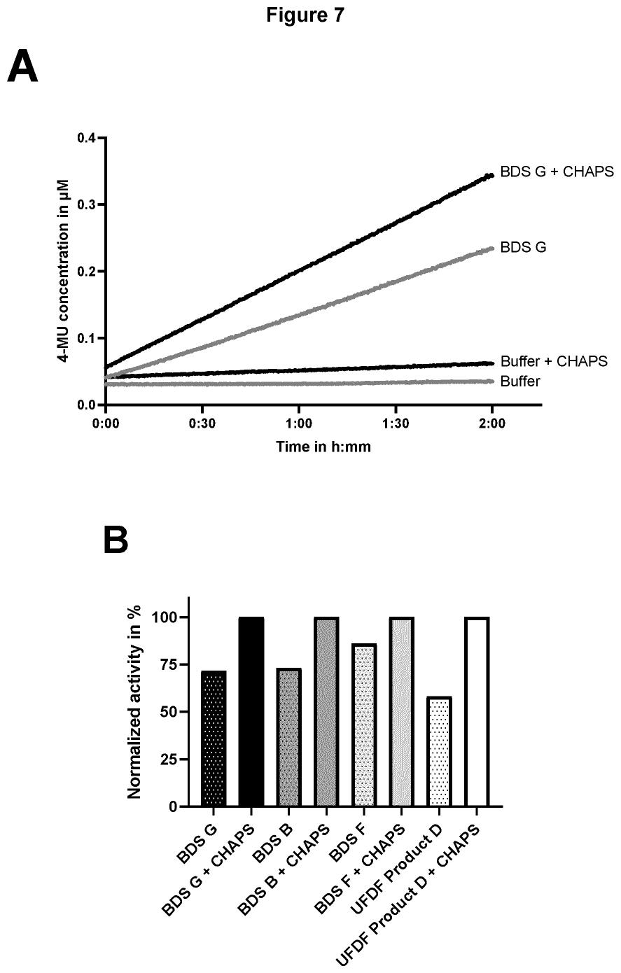

First, a surfactant was tested above its CMC (critical micelle concentration). Therefore 10 mM CHAPS were added to the assay. Specifically, the assay was performed with 30 μM 4-MUD in AMT assay buffer (75 mM acetate, 75 mM MES, 150 mM Tris, 150 mM NaCl, pH 5.5) with or without 10 mM CHAPS using a microplate reader (λₑₘ=450 nm, λₑₓ=330 nm, bottom read mode) in a black 96 well plate. The samples tested were drug substance samples comprising Product G, Product B, Product F and a sample following ultrafiltration/diafiltration of Product D, each added at 72 μL per well. The results are shown in FIGS. 7A and B, demonstrating that the presence of a surfactant, such as CHAPS above its CMC increased assay sensitivity. Without being bound by theory it is hypothesized that a surfactant creates an environment that promotes lipase activity by allowing the rearrangement and opening of the lid or flap, which has been described to cover the active site (Grochulski P, Li Y, Schrag J D, et al. Protein Sci 1994; 3:82-91 and Grochulski P, Bouthillier F, Kazlauskas R J, et al. Biochemistry 1994; 33:3494-500). Therefore, a concentration of 10 mM CHAPS in the final reaction-mix was selected. As a consequence, lipase hydrolysis was increased as well as the sensitivity of the assay (FIG. 7).

The surfactant can be another surfactant, but is required to be a mild and particularly a non-denaturing surfactant to maintain the native structure and activity of the proteins with lipase activity. Furthermore, it is important that the surfactant does not compete with or otherwise inhibit lipases/hydrolyses. CHAPS does not exhibit an ester bond or an acyl chain and is thus not a substrate for lipases. It therefore does not compete with the substrate and hence does not affect sensitivity of the assay. Additionally, CHAPS mediates solubility of 4-MUD in water in the used concentrations (FIG. 3).

### Example 5: Measuring Samples with Different pH Values

The activity of enzymes is often influenced by pH. As the expected pH range of in-process control (IPC) samples reaches from 3.5 to 7.5 (Table 1) an influence on the observed activity of contaminating lipases was expected. To maximize comparability between samples of different pH values, first buffer components were identified to maintain the pH constant at 7.4 in the reaction mixture. Table 1 summarizes different IPC samples from one downstream process to demonstrate the pH of the sample originating from different purification steps. Shown is the pH of each sample and the corresponding pH of the reaction mixture. Similar pH variations were found for different antibodies or Fc-fusion proteins during downstream processing. The experiment was carried out with several products that provided similar results.

TABLE 1

| Process stage | pH of the sample | pH in final mix |
|---|---|---|
| Harvested Cell Culture Fluid | 7.3 | 7.4 |
| (HCCF) | | |
| Protein A | 4.2 | 7.4 |
| Virus Inactivation (VI) | 3.5 | 7.4 |
| Depth Filtration (DF) | 5.5 | 7.4 |
| Anion Exchange | 7.5 | 7.4 |
| Chromatopgraphy (AIEX) | | |
| Cation Exchange | 5.5 | 7.4 |
| Chromatography(CIEX) | | |
| Virus Filtration (VF) | 5.5 | 7.4 |
| Ultra-/Diafiltration (UF/DF) | 5.5 | 7.4 |
| Bulk Drug Substance (BDS) | 5.5 | 7.4 |

Analysing the IPC samples at the same pH maintains constant reaction conditions and hence increases comparability of resulting data. However, in some cases it is beneficial to alter the pH, e.g. to find conditions that reduce hydrolytic activity (see e.g., FIG. 2) and support formulation development. Therefore, the three-component AMT Buffer System was developed.

### Example 6: Influence of Ionic Strength

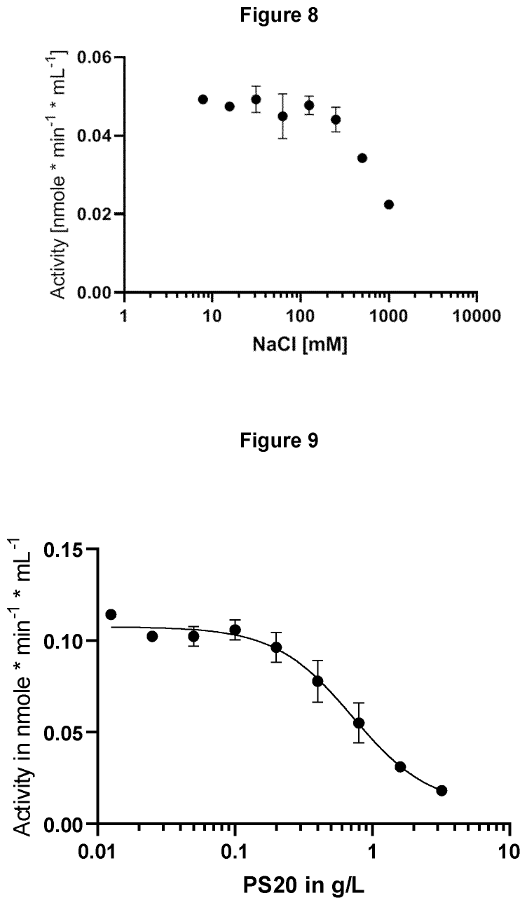

In order to prevent protein aggregation in samples and to maintain the native structure of proteins, it is beneficial to provide ions. However, the ionic strength may also affect enzyme activity. Therefore, the influence of the ionic strength on the measured hydrolytic activity was tested. Hydrolytic activity of an exemplary drug product sample was measured in the presence of varying concentrations of NaCl (1000 mM-7.8125 mM). All measurements were performed with 30 μM 4-MUD in AMT assay buffer without NaCl (75 mM acetate, 75 mM MES, 150 mM Tris, 10 mM CHAPS, pH 5.5) and 72 μL sample using a microplate reader (λₑₘ=450 nm, λₑₓ=340 nm, 25° C., top read mode) in a black 96 well plate. The dependence of hydrolytic activity to ionic strength has been investigated with several mAbs and is exemplarily shown for Product F in FIG. 8.

As may be taken from FIG. 8 lipase activity decreased at a NaCl concentration of 250 mM or higher. For the AMT buffer a NaCl concentration of 150 mM was therefore found to be optimal.

### Example 7: Inhibition of the Lipase Assay by Polysorbate

Polysorbate in the final drug product is likely to function as a competitive substrate for the traceable fluorogenic substrate (4-MUD). Polysorbate 20 (PS20) or polysorbate 80 concentrations in a drug product may range between about 0.2 to 1.0 g/L and are typically in the range of 0.2 to 0.4 g/L PS20 or PS80 or a mixture thereof. Therefore, an experiment was set up using ultrafiltration-diafiltration material of an antibody as active pharmaceutical ingredient (API in water, without PS20) and adding varying concentrations of PS20 (0.0125-3.2 mg/mL) to the reaction mixture.

4-MUD hydrolytic activity in the samples was measured in the presence of 0.0125-3.2 mg/ml PS20 (final reaction mixture concentration). All measurements were performed with 30 μM 4-MUD in AMT assay buffer (50 mM acetate, 50 mM MES, 100 mM Tris, 150 mM NaCl, 10 mM CHAPS, pH 5.5) using a microplate reader (λₑₘ=450 nm, λₑₓ=330 nm, top read mode) in a black 96 well plate. In this initial experiment, the AMT buffer was used at a lower concentration, which later turned out to be too low for buffering at pH 4 and pH 8. Therefore, the concentrations were increased to the concentrations as described above.

The results indicate that PS20 is a competitive inhibitor to 4-MUD hydrolysis at high concentration. No inhibition was observed up to 0.2 g/L PS20 in the reaction mixture (≥0.8 g/L in DP), while a concentration-dependent inhibition was observed at concentrations of 0.4 g/L or higher in the assay sample, 4-MUD hydrolysis was clearly still detectable at concentrations of 0.4 g/L PS20 (FIG. 9). By contrast Jahn et al., (Pharm. Res., 2020, 37:118, pages 1-13) already observed almost complete inhibition at 0.02% PS20 (w/v) (0.2 g/L) in the sample, indicating the higher sensitivity of the lipase assay according to the present invention.

Inhibition of the lipase assay was expected as this indicates a correlation between the 4-MUD hydrolysis activity detected in the assay and polysorbate degradation. However, in order to be able to determine lipase activity also in the drug product, it is advantageous if concentrations typically applied in antibody formulation, such as 0.1 to 0.4 g/L do not or only slightly interfere with the assay.

Overall, the data show that no significant influence on the activity is expected for 4-MUD experiments analyzing samples comprising polysorbate concentrations commonly used in formulated drug substance.

### Example 8: Effects of Other Detergents on Lipase Activity

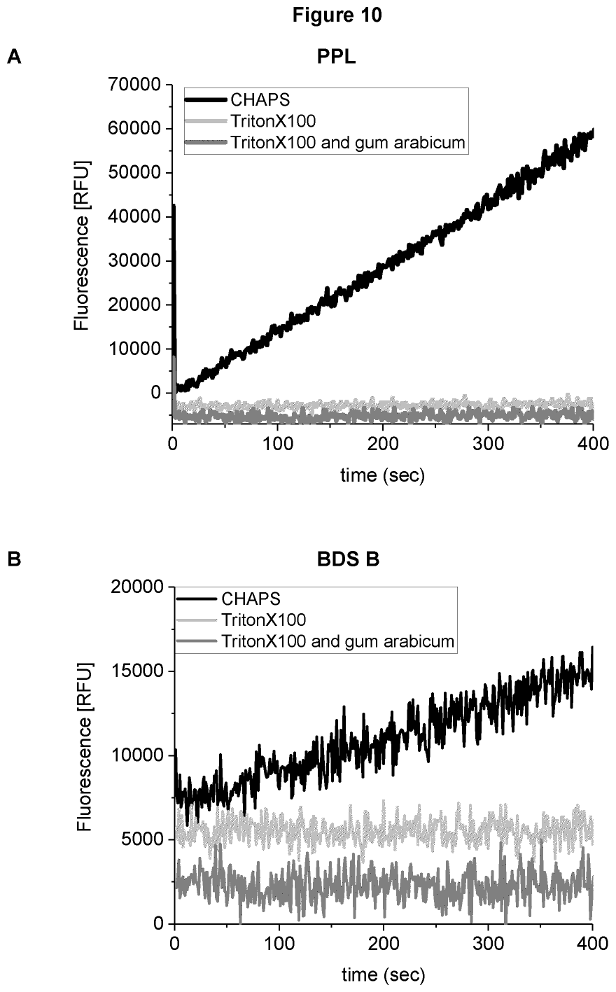

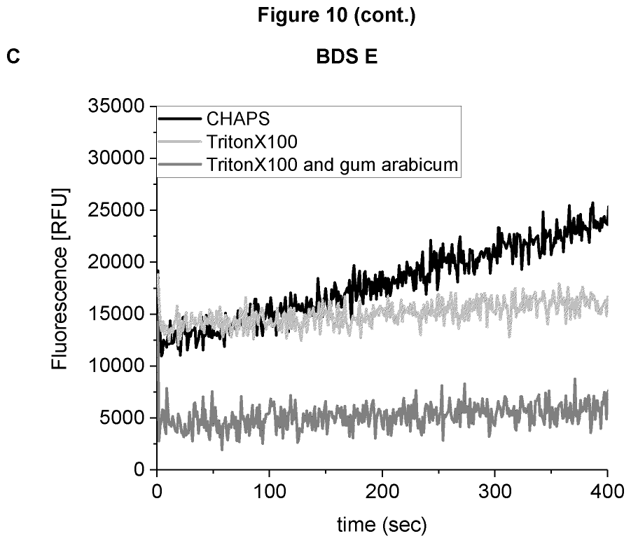

Jahn et al., (Pharm. Res., 2020, 37:118, pages 1-13) reported a de-activating effect on PPL activity caused by the presence of surfactants, amongst them Triton X-100. Therefore, hydrolytic activity of PPL (0.024 mg/ml in the reaction mixture) and in BDS B and BDS E (2.4 mg/ml in the reaction mixture) was measured in ATM buffer, pH 5.5, with either 10 mM CHAPS, 0.25% Triton X-100 or 0.25% Triton X-100 and 0.125% gum arabicum. The results shown in FIG. 10A-C demonstrate that CHAPS outperforms Triton X-100 and Triton X-100 and gum arabicum. Gum arabicum is an emulsifier and no effect of gum arabicum on the activity has been observed in any of the experiments.

### Example 9: Suitability of the 4-MUD Assay for IPC Sample Analysis

Suitability of the 4-MUD assay for IPC sample analysis was tested on some exemplary processes. Therefore, in-process control samples from different downstream process steps were analyzed as to whether the solubility is sufficient, the pH is in the expected range and the kinetics measurement meets the requirements (pseudo-zero order reaction rate).

Table 2 shows the applicability of process samples with regard to the possible influence of particles, ion strength, pH and several other influence factors. Samples were analysed in triplicates and reactions mixtures that did not meet the requirements of pseudo-zero order reaction rate were excluded from analysis.

TABLE 2
Suitability of mAb IPC samples in the Assay (see Table 1 for abbreviations)

| | HCCF | Protein A | VI | DF | AIEX | CIEX | VF | UF/DF | BDS |
|---|---|---|---|---|---|---|---|---|---|
| Product F | — | AWL | AWL | AWL | — | AWL | AWL | AWL | AWL |
| Product B | — | AWL | AWL | AWL | AWL | AWL | — | AWL | AWL |
| Product G | — | AWL | — | AWL | AWL | AWL | — | AWL | AWL |

Blank (—): data not available
Filled (“AWL”): applicable without limitations

Suitability of the assay could be shown and, as a result, the assay was applicable in all process steps tested including following ultra-filtration/diafiltration (UF/DF) and in the final formulated bulk drug substance (BDS).

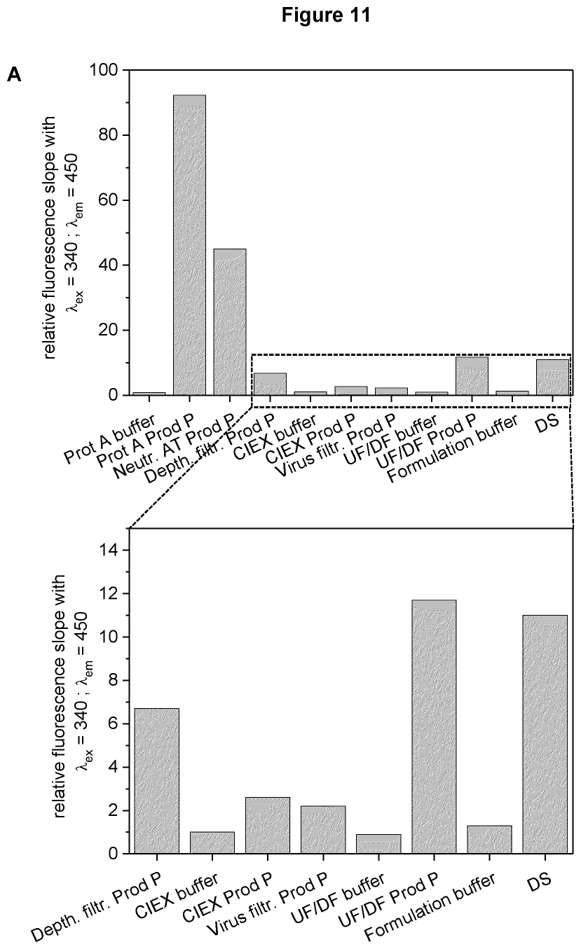

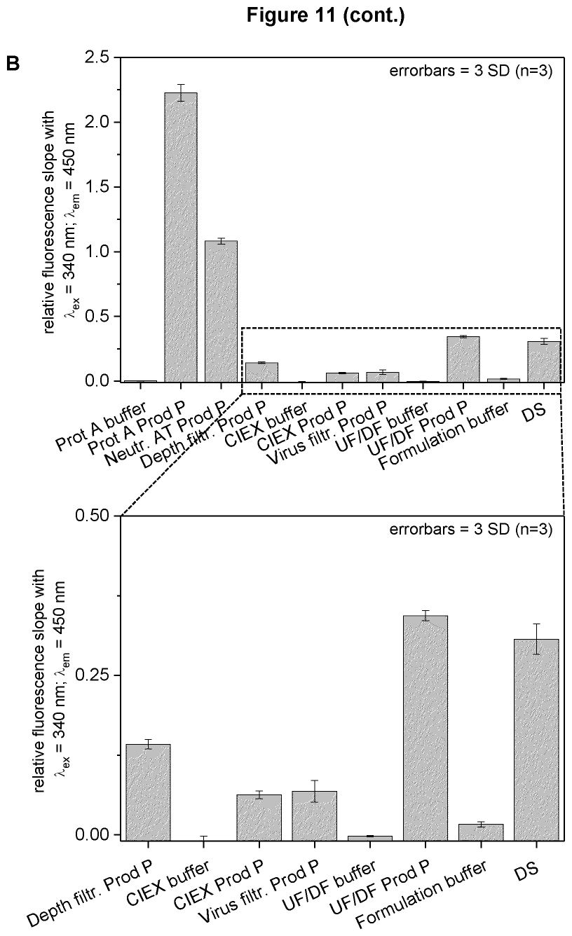

This has further been determined for a specific antibody using the lipase assay in a spectrometer (FIG. 11A) and in a plate reader (FIG. 11B).

The results of the 4-MUD plate reader assay generally correlated well with the results of the spectrometer. Both read outs were able to demonstrate low lipase activity in the product containing sample compared to the elution buffer alone as well as in the drug substance compared to formulation buffer alone. The 4-MUD assay can be performed in a microtiter plate format for high-throughput purposes and can therefore be automated.

### Example 10: Effect of a Lipase Inhibitor

There are several substances available to inhibit enzymatic hydrolysis of ester bonds. Consequently, an inhibitor capable of reducing polysorbate degradation in drug product, drug substance and other process steps of biopharmaceutical development should also inhibit the hydrolytic activity monitored by 4-MUD assay.

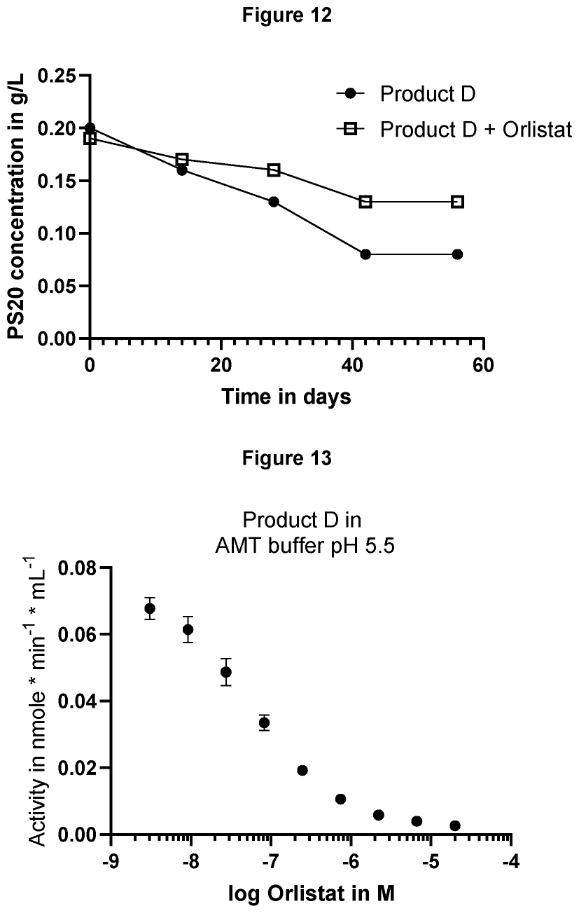

Therefore, samples were incubated with or without 1 μM Orlistat—an irreversible lipase inhibitor (Borgström 1988)—and tested for stability at room temperature (˜22° C.) for 2 months at pH 5.5. A drug product sample (Product D) with 0.2 mg/mL PS20 was incubated at RT with several pull points up to 56 days. PS20 content was measured at indicated time points using a HPLC-CAD method. As shown in FIG. 12, 1 μM Orlistat resulted in a reduced degradation of PS20 compared to the control reaction (DMSO only).

The hydrolytic activity of the same drug product sample (Product D) was measured in the presence of varying concentrations of Orlistat (7.3 nM-20 μM) using the lipase assay. All measurements were performed with 30 μM 4-MUD in AMT assay buffer (75 mM acetate, 75 mM MES, 150 mM Tris, 150 mM NaCl, 10 mM CHAPS, pH 5.5) using a microplate reader (λₑₘ=450 nm, λₑₓ=330 nm, top read mode) in a black 96 well plate. As shown in FIG. 13, Orlistat inhibited lipase activity in a concentration dependent manner as determined by the 4-MUD assay. The results suggest that Orlistat is inhibiting hydrolytic activity responsible for Polysorbate degradation (FIG. 12) as well as the hydrolytic activity monitored using the 4-MUD assay (FIG. 13). Moreover, inhibition was detectable within minutes rather than days using the HPLC-CAD method and at much lower inhibitor concentrations.

### Example 11: Polysorbate Degradation in Spiking Experiments Compared with 4-MUD Activity

Polysorbate spiking experiments were carried out to test for a correlation between hydrolytic activity as measured by 4-MUD Assay with polysorbate degradation. To assess Polysorbate degradation rate, relevant in-process-steps (IPC) samples were spiked with polysorbate and consequently, the polysorbate content was monitored over time by fluorescence micelle assay (FMA). The IPC samples tested include samples following Protein A purification (MabSelect), following depth filtration (Cuno), ion exchange chromatography (Poros) and bulk drug substance (BDS).

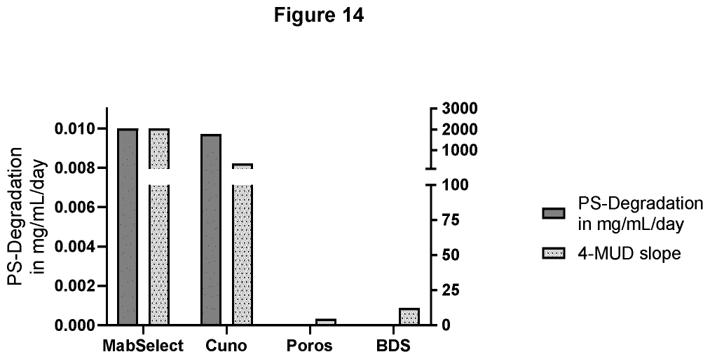

Polysorbate degradation was determined using a FMA assay and compared to the hydrolytic activity as measured by the lipase assay using a phosphate assay buffer (81 mM Na₂HPO₄, 19 mM NaH₂PO₄, 140 mM NaCl, 10 mM CHAPS, pH 7.4) containing 3 μM 4-MUD using a fluorescent spectrometer (λₑₘ=450 nm, λₑₓ=340 nm) in a 1 cm macro-cuvette. The results (FIG. 14) suggest a correlation of the hydrolytic activity as measured by the lipase assay with polysorbate degradation in relevant IPC steps of product B.

1. A method for detecting lipase activity in a sample comprising a recombinant protein comprising
(a) providing at least one sample comprising a recombinant protein produced in a eukaryotic cell;
(b) contacting the at least one sample with a reaction solution to form a reaction mixture, wherein the reaction solution comprises:
(i) a buffer having a pH of about pH 4 to about pH 9,
(ii) a non-denaturing surfactant not having an ester-bond, wherein the surfactant is a non-ionic or zwitter-ionic surfactant,
(iii) a substrate comprising the chromophore 4-methylumbelliferyl (4-MU) in the form of a 4-MU ester, wherein the 4-MU ester is a saturated unbranched-chain fatty acid (C6-C16) 4-MU ester, and
(iv) optionally a non-buffering salt;
(c) incubating the sample and the substrate in the reaction mixture;
(d) detecting lipase activity by measuring hydrolysis of the 4-MU ester and detecting the fluorescence intensity of the released chromophore 4-MU; optionally measuring hydrolysis by detecting the fluorescence intensity of the released chromophore 4-MU over time, while incubating the sample and the substrate in the reaction mixture according to step (c).

2. The method of claim 1, wherein the sample and the substrate in the reaction mixture are incubated for any time period between 2 min and less than 5 hours.

3. The method of claim 1, wherein the substrate is selected from the group consisting of 4-methylumbelliferyl octanoate, 4-methylumbelliferyl nonanoate, 4-methylumbelliferyl decanoate (4-MUD), 4-methylumbelliferyl undecanoate and 4-methylumbelliferyl dodecanoate.

4. The method of claim 1, wherein
(a) the surfactant has a final concentration in the reaction mixture above its critical micelle concentration in the reaction mixture; and/or
(b) the surfactant
(i) is selected from the group consisting of CHAPS, CHAPSO and Zwittergent; or
(ii) is CHAPS and is provided at a final concentration in the reaction mixture of about 8 mM to about 20 mM; or
(iii) is not polyethylene glycol tert-octylphenyl ether (Triton X-100) and not polyethylene glycol nonylphenyl ether (NP-40).

5. The method of claim 1, wherein the buffer comprises one or more buffer substances selected from the group consisting of a formic acid, acetic acid, lactic acid, citric acid, malic acid, maleic acid, glycine, glycylglycine, succinic acid, TES (2-{[tris(hydroxymethyl)methyl]amino}ethanesulfonic acid), MOPS (3-(N-morpholino)propanesulfonic acid), PIPES (piperazine-N,N′-bis(2-ethanesulfonic acid)), MES (2-(N-morpholino)ethanesulfonic acid), Tris base, Tris, Bis-Tris, Bis-Tris-Propane, Bicine (N,N-bis(2-hydroxyethyl)glycine), HEPES (4-2-hydroxyethyl-1-piperazineethanesulfonic acid), TAPS (3-([tris(hydroxymethyl)methyl]amino}pro-panesulfonic acid), Tricine (N-tris(hydroxymethyl)methylglycine), Na₂HPO₄ and NaH₂PO₄.

6. The method of claim 1, wherein the buffer
(a) has a pH of about 5 to about 7.5; and/or
(b) is a multi-component buffer having a buffering range from at least about pH 5 to at least about pH 7.5.

7. The method of claim 1, wherein the reaction solution comprises a non-buffering salt and:
(a) the non-buffering salt is selected from the group consisting of NaCl, KCl and CaCl₂; and/or
(b) the non-buffering salt has a concentration of about 100 mM to about 200 mM in the reaction mixture; and/or
(c) the ionic strength of non-buffering salt is about 200 mM or less in the reaction mixture in the reaction mixture; and/or
(d) the cumulative ionic strength of the buffer and the non-buffering salt in the reaction mixture is about 450 mM or less.

8. The method of claim 1, wherein
(a) the at least one sample is a harvested cell culture fluid (HCCF), an in-process control (IPC) sample, a drug substance sample or a drug product sample; and/or
(b) the recombinant protein is not a lipase and/or an enzyme having lipase activity; and/or
(c) the recombinant protein is selected from the group consisting of an antibody, an antibody fragment, an antibody derived molecule and a fusion protein.

9. A method of manufacturing a recombinant protein of interest comprising the steps of
(i) cultivating a eukaryotic cell expressing a recombinant protein of interest in cell culture;
(ii) harvesting the recombinant protein;
(iii) purifying the recombinant protein; and
(iv) optionally formulating the recombinant protein into a pharmaceutically acceptable formulation suitable for administration; and
(v) obtaining at least one sample comprising the recombinant protein in steps (ii), (iii) and/or (iv);
wherein the method further comprises detecting lipase activity in a sample obtained in step (v) comprising:
(a) providing the at least one sample;
(b) contacting the at least one sample with a reaction solution to form a reaction mixture, wherein the reaction solution comprises:
(i) a buffer having a pH of about pH 4 to about pH 9,
(ii) a non-denaturing surfactant not having an ester-bond, wherein the surfactant is a non-ionic or zwitter-ionic surfactant,
(iii) a substrate comprising the chromophore 4-methylumbelliferyl (4-MU) in the form of a 4-MU ester, wherein the 4-MU ester is a saturated unbranched-chain fatty acid (C6-C16) 4-MU ester, and
(iv) optionally a non-buffering salt;
(c) incubating the sample and the substrate in the reaction mixture; and
(d) detecting lipase activity by measuring hydrolysis of the 4-MU ester and detecting the fluorescence intensity of the released chromophore 4-MU; optionally measuring hydrolysis by detecting the fluorescence intensity of the released chromophore 4-MU over time, while incubating the sample and the substrate in the reaction mixture according to step (c).

10. The method of claim 9, comprising obtaining at least one sample comprising the recombinant protein in
step (ii), wherein the sample is a harvested cell culture fluid (HCCF) or a cell lysate;
step (iii), wherein the sample is an in-process control (IPC) sample; and/or
step (iv), wherein the sample is a drug substance sample or a drug product sample.

11. The method of claim 1, wherein the lipase activity detected in the at least one sample is contaminating lipase activity.

12. A kit for determining contaminating lipase activity in a sample comprising a recombinant protein comprising:
(i) a buffer having a pH of about pH 4 to about pH 9;
(ii) a non-denaturing surfactant not having an ester-bond, wherein the surfactant is a non-ionic or zwitter-ionic surfactant;
(iii) a substrate comprising the chromophore 4-methylumbelliferyl (4-MU) in the form of a 4-MU ester, wherein the substrate is a saturated unbranched-chain fatty acid (C6 to C16) 4-MU ester; and
(iv) optionally a non-buffering salt; and/or
(v) optionally water for dilution.

13. The kit of claim 12, wherein
(a) the substrate is selected from the group consisting of 4-methylumbelliferyl octanoate, 4-methylumbelliferyl nonanoate, 4-methylumbelliferyl decanoate (4-MUD), 4-methylumbelliferyl undecanoate and 4-methylumbelliferyl dodecanoate; and/or
(b) the kit further comprises an organic solvent for dissolving the substrate.

14. The kit of claim 12, wherein
(a) the surfactant is selected from the group consisting of CHAPS, CHAPSO, Zwittergent and a saponin; and/or
(b) the surfactant is not polyethylene glycol tert-octylphenyl ether (Triton X-100) and not polyethylene glycol nonylphenyl ether (NP-40);
(c) the buffer comprises one or more buffer substances selected from the group consisting of a formic acid, acetic acid, lactic acid, citric acid, malic acid, maleic acid, glycine, glycylglycine, succinic acid, TES (2-{[tris(hydroxymethyl)methyl]amino}ethanesulfonic acid), MOPS (3-(N-morpholino)propanesulfonic acid), PIPES (piperazine-N,N′-bis(2-ethanesulfonic acid)), MES (2-(N-morpholino)ethanesulfonic acid), Tris base, Tris, Bis-Tris, Bis-Tris-Propane, Bicine (N,N-bis(2-hydroxyethyl)glycine), HEPES (4-2-hydroxyethyl-1-piperazineethanesulfonic acid), TAPS (3-([tris(hydroxymethyl)methyl]amino}pro-panesulfonic acid), Tricine (N-tris(hydroxymethyl)methylglycine), Na₂HPO₄ and NaH₂PO₄.

15. The kit of claim 12, wherein
(a) the buffer has a pH of about 5 to about 7.5, preferably the buffer has a pH of about 5.5 to about 7.5;
(b) the buffer is a multi-component buffer having a buffering range from at least about pH 5 to at least about pH 7.5, preferably from at least about pH 4 to at least about pH 8; and/or
(c) the non-buffering salt is selected from the group consisting of NaCl, KCl and CaCl₂), preferably wherein the non-buffering salt is NaCl or KCl.

16. The kit of claim 12, wherein
(a) the kit further comprises one or more microtiter plate having 96 wells or a multiple of 96 wells; and/or
(b) the buffer, the surfactant and the optional non-buffering salt are premixed as an assay buffer that is about 3 to about 5-fold concentrated relative to a final reaction mixture and/or provided as a dry mixture.

17. The method of claim 10, wherein the lipase activity detected in the at least one sample is contaminating lipase activity.
